(19) United States
(12) Patent Application Publication
(10) Pub. No.: US 2023/0131429 A1
(43) Pub. Date: Apr. 27, 2023

(54) PHOTO-DECOMPOSABLE COMPOUND, PHOTORESIST COMPOSITION INCLUDING THE SAME, AND METHOD OF MANUFACTURING INTEGRATED CIRCUIT DEVICE

(71) Applicant: SAMSUNG ELECTRONICS CO., LTD., Suwon-si (KR)

(72) Inventors: Jiyup KIM, Suwon-si (KR); Hyunwoo KIM, Seongnam-si (KR); Hyun-Ji SONG, Anyang-si (KR); Juyoung KIM, Hwaseong-si (KR); Jinjoo KIM, Seoul (KR); Sunghwan PARK, Suwon-si (KR); Giyoung SONG, Asan-si (KR)

(21) Appl. No.: 17/730,532

(22) Filed: Apr. 27, 2022

(30) Foreign Application Priority Data
Oct. 19, 2021 (KR) 10-2021-0139118

(51) Int. Cl. G03F 7/004; C07C 381/12; C07C 309/12; H01L 21/027

(52) U.S. Cl. CPC G03F 7/0045; C07C 381/12; C07C 309/12; H01L 21/0275; C07C 2603/74

(57) ABSTRACT

A photo-decomposable compound, a photoresist composition including the photo-decomposable compound, and a method of manufacturing an integrated circuit (IC) device using the photoresist composition, the photo-decomposable compound including a phenyl sulfonium cation component; and an anion component, wherein the phenyl sulfonium cation component has a protecting group, which is decomposable by an action of acid to generate an alkali-soluble group in response to exposure, the anion component generates acid in response to exposure, the protecting group is represented by *—C(═O)OR, in which R is a substituted or unsubstituted t-butyl group or a substituted or unsubstituted C3 to C30 alicyclic group, and * is a bonding site, and the protecting group is bonded to a phenyl group of the phenyl sulfonium cation component through an ether linking group.

### CROSS-REFERENCE TO RELATED APPLICATION

This application is based on and claims priority under 35 U.S.C. § 119 to Korean Patent Application No. 10-2021-0139118, filed on Oct. 19, 2021, in the Korean Intellectual Property Office, the disclosure of which is incorporated by reference herein in its entirety.

### BACKGROUND

### 1. Field

Embodiments relate to a photo-decomposable compound, a photoresist composition including the same, and a method of manufacturing an integrated circuit (IC) device.

### 2. Description of the Related Art

As IC devices have rapidly been downscaled and highly integrated, techniques for ensuring the dimensional precision of a pattern to be formed when the pattern is formed using a photolithography process have been considered.

### SUMMARY

The embodiments may be realized by providing a photo-decomposable compound including a phenyl sulfonium cation component; and an anion component, wherein the phenyl sulfonium cation component has a protecting group, which is decomposable by an action of acid to generate an alkali-soluble group in response to exposure, the anion component generates acid in response to exposure, the protecting group is represented by *—C(═O)OR, in which R is a substituted or unsubstituted t-butyl group or a substituted or unsubstituted C3 to C30 alicyclic group, and * is a bonding site, and the protecting group is bonded to a phenyl group of the phenyl sulfonium cation component through an ether linking group.

The embodiments may be realized by providing a photoresist composition including a chemically amplified polymer; a photo-decomposable compound including a phenyl sulfonium cation component and an anion component, the phenyl sulfonium cation component having a protecting group that is decomposable by an action of acid in response to exposure and generates an alkali-soluble group in response to exposure, and the anion component generating an acid in response to exposure; and a solvent, wherein the photo-decomposable compound is represented by Formula 1:

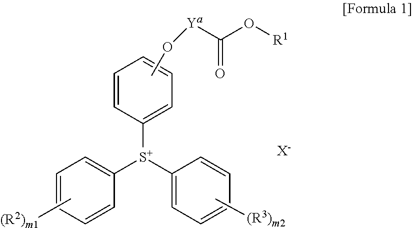

in Formula 1, R¹ is a substituted or unsubstituted t-butyl group or a substituted or unsubstituted C3 to C30 alicyclic group, R² and R³ are each independently a hydrogen (H) atom, an iodine (I) atom, a fluorine (F) atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom, provided that at least one of R² and R³ is an iodine (I) atom, a fluorine (F) atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom, R² and R³ are each independently bonded on Formula 1 at a single location or bonded at multiple locations to form a ring along with another atom included in Formula 1, m1 and m2 are each independently an integer ranging from 0 to 3, provided that at least one of m1 and m2 is 1, 2, or 3, Y^{a} is a substituted or unsubstituted C1 to C30 divalent linear or cyclic group, and X⁻ is a monovalent anion.

The embodiments may be realized by providing a method of manufacturing an integrated circuit (IC) device, the method including forming a photoresist film on a underlayer film by using a photoresist composition that includes a chemically amplified polymer, a photo-decomposable compound, and a solvent, the photo-decomposable compound including a phenyl sulfonium cation component and an anion component, the phenyl sulfonium cation component having a protecting group that is decomposable by an action of acid in response to exposure and generates an alkali-soluble group in response to exposure, and the anion component generating acid in response to exposure, the protecting group being represented by *—C(═O)OR, in which R is a substituted or unsubstituted t-butyl group or a substituted or unsubstituted C3 to C30 alicyclic group, and * is a bonding site, and the protecting group being bonded to a phenyl group of the phenyl sulfonium cation component through an ether linking group, exposing a first area, which is a portion of the photoresist film, to generate acid from the anion component of the photo-decomposable compound, deprotect an acid-labile group included in the chemically amplified polymer, and generate an alkali-soluble group from the phenyl sulfonium cation component of the photo-decomposable compound in the first area; removing the exposed first area of the photoresist film and the alkali-soluble group using a developer to form a photoresist pattern, the photoresist pattern comprising a non-exposed area of the photoresist film, and processing the underlayer film using the photoresist pattern.

### BRIEF DESCRIPTION OF THE DRAWINGS

Features will be apparent to those of skill in the art by describing in detail exemplary embodiments with reference to the attached drawings in which:

FIG. 1 is a flowchart of a method of manufacturing an integrated circuit (IC) device, according to embodiments;

FIGS. 2A to 2E are cross-sectional views of stages in a method of manufacturing an IC device, according to embodiments;

FIG. 3 shows critical dimension-scanning electron microscope (CD-SEM) images of photoresist patterns obtained using a compound according to Examples and photoresist patterns obtained using a Comparative Compound; and

FIG. 4 is a graph showing evaluation results of underdose margins in the cases of using compounds according to Examples and the cases of using Comparative Compounds.

### DETAILED DESCRIPTION

A photo-decomposable compound according to embodiments may include, e.g., a phenyl sulfonium cation component (which may be used interchangeably with the term ‘cation component’ herein) and an anion component. The phenyl sulfonium cation component may have a protecting group, which may decompose by an action of acid during or in response to exposure and may generate an alkali-soluble group, and the anion component may generate acid due or in response to exposure.

In an implementation, the protecting group may have a structure of the following Structural Formula 1:

*—C(═O)OR [Structural Formula 1]

In Structural Formula 1, R may be or may include, e.g., a substituted or unsubstituted t-butyl group or a substituted or unsubstituted C3 to C30 alicyclic group, and * is a bonding site. In an implementation, the protecting group having the Structural Formula 1 may be bonded to a phenyl group of the cation component through an ether linking group. As used herein, the term “or” is not an exclusive term, e.g., “A or B” would include A, B, or A and B.

As used herein, unless otherwise defined, the term “substituted” may refer to including at least one sub substituent, e.g., a halogen atom (e.g., a fluorine (F) atom, a chlorine (C1) atom, a bromine (Br) atom, or an iodine (I) atom), hydroxyl, amino, thiol, carboxyl, carboxylate, ester, amide, nitrile, sulfide, disulfide, nitro, C1 to C20 alkyl, C1 to C20 cycloalkyl, C2 to C20 alkenyl, C1 to C20 alkoxy, C2 to C20 alkenoxy, C6 to C30 aryl, C6 to C30 aryloxy, C7 to C30 alkylaryl, or a C7 to C30 alkylaryloxy group.

In the protecting group having the structure of Structural formula 1, which is included in the photo-decomposable compound according to the embodiments, R may have a structure that is substituted with a first substituent. In an implementation, R may include a t-butyl group substituted with the first substituent or a C3 to C30 tertiary alicyclic group substituted with the first substituent. In an implementation, the first substituent may include, e.g., a halogen atom, a hydroxyl group, a C1 to C10 alkyl group, a C1 to C10 alkoxy group, a C1 to C10 halogenated alkyl group, an unsubstituted C6 to C30 aryl group, or a C6 to C30 aryl group in which some of carbon atoms included in the first substituent are substituted with a halogen atom or a hetero atom-containing group. The halogen atom that may be included in the first substituent may be a fluorine atom, a chlorine atom, a bromine atom, or an iodine atom. The halogenated alkyl group may include at least one halogen atom selected from the F atom, the chlorine atom, the bromine atom, and the iodine atom. The hetero atom may be an oxygen atom, a sulfur atom, or a nitrogen atom. In an implementation, the hetero atom-containing group may be, e.g., —O—, —C(═O)—O—, —O—C(═O)—, —C(═O)—, —O—C(═O)—O—, —C(═O)—NH—, —NH—, —S(═O)₂—, or —S(═O)₂—O—.

In an implementation, the photo-decomposable compound according to the embodiments may be, e.g., represented by Formula 1.

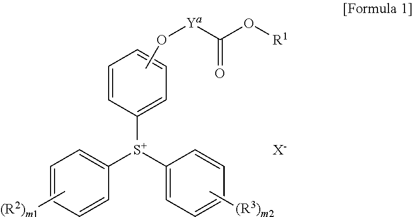

In Formula 1, le may be or may include, e.g., a substituted or unsubstituted t-butyl group or a substituted or unsubstituted C3 to C30 alicyclic group.

R² and R³ may each independently be or include, e.g., a hydrogen atom, an iodine atom, a fluorine atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom. In an implementation, R² and R³ may each independently be bonded on Formula 1 at a single location or may be bonded at multiple locations to form a ring along with another atom included in Formula 1. In an implementation, at least one of R² and R³ may not be a hydrogen (H) atom, e.g., may be or may include an iodine atom, a fluorine atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom.

m1 and m2 may each independently be, e.g., an integer ranging from 0 to 3. In an implementation, at least one of m1 and m2 may be, e.g., 1, 2, or 3.

Y^{a} may be or may include, e.g., a substituted or unsubstituted C1 to C30 divalent linear or cyclic group.

X⁻ may be, e.g., a monovalent anion.

In an implementation 1, R¹ may have the same structure as R of Structural Formula 1. In an implementation, in Formula 1, R¹ may be, e.g., a substituted or unsubstituted t-butyl group or a substituted or unsubstituted C3 to C30 tertiary alicyclic group.

In an implementation, R¹ may include, e.g., a tertiary alicyclic group including an alicyclic hydrocarbon group, and the alicyclic hydrocarbon group may include a group in which two hydrogen atoms are excluded from C3 to C30 monocycloalkane. In an implementation, R¹ may include, e.g., a tertiary alicyclic group including an alicyclic hydrocarbon group, and the alicyclic hydrocarbon group may include a group in which two hydrogen atoms are excluded from C7 to C12 polycycloalkane. In an implementation, R¹ may include, e.g., an organic group including a C6 to C30 aromatic ring including at least one substituent selected from a C5 to C7 aromatic hydrocarbon group and derivatives thereof, a fluorine atom, and a hydroxyl group or an organic group including a C6 to C30 heteroaromatic ring including at least one substituent selected from a fluorine atom and a hydroxyl group.

In an implementation, in Formula 1, R¹ may have, e.g., one of the following structures.

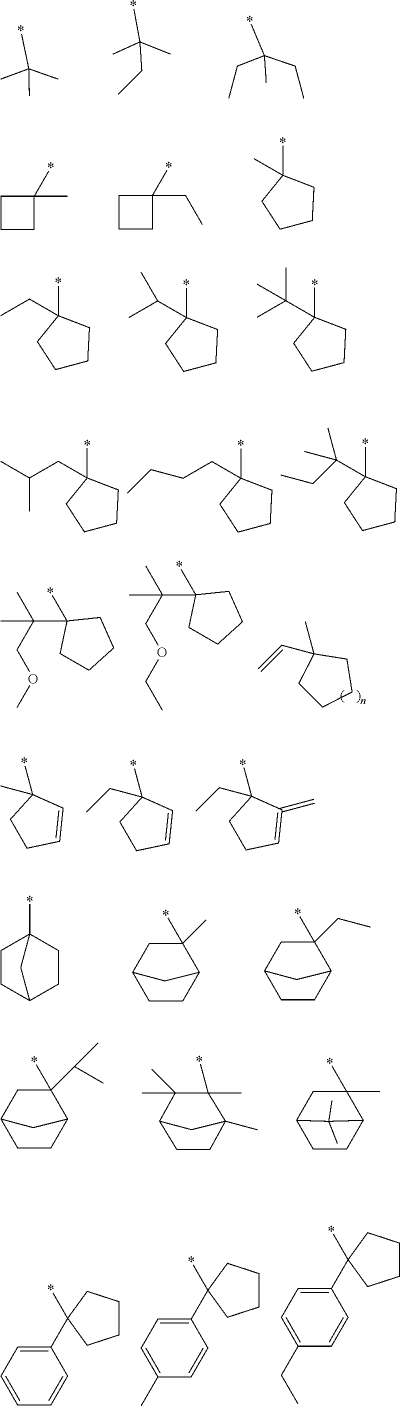

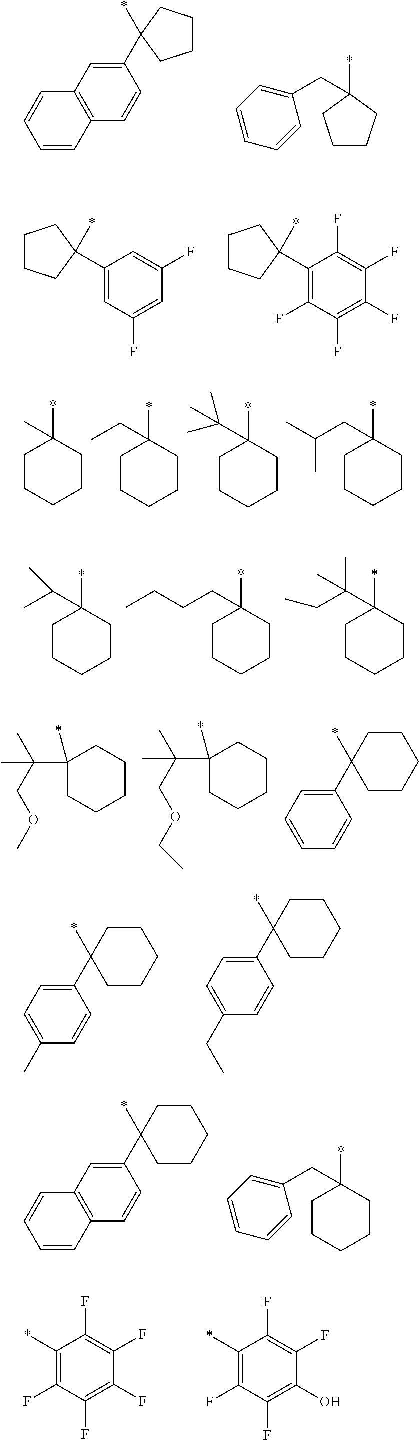

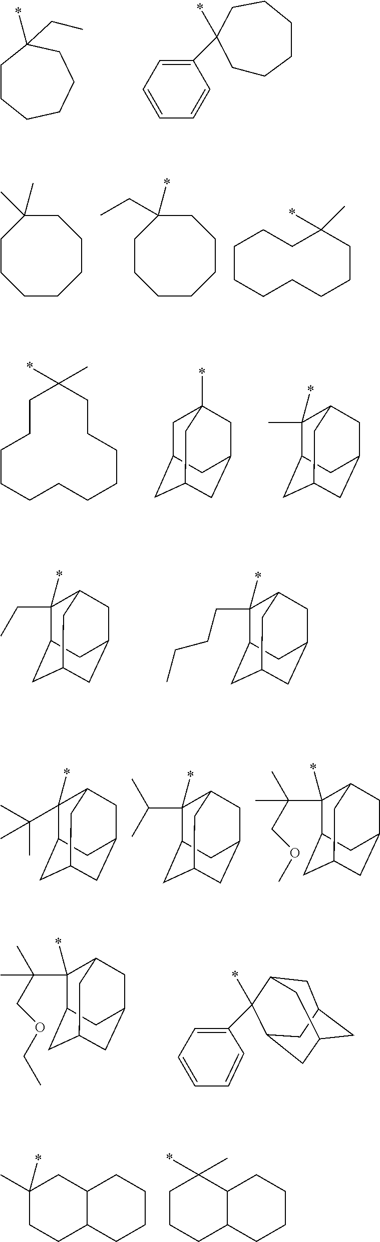

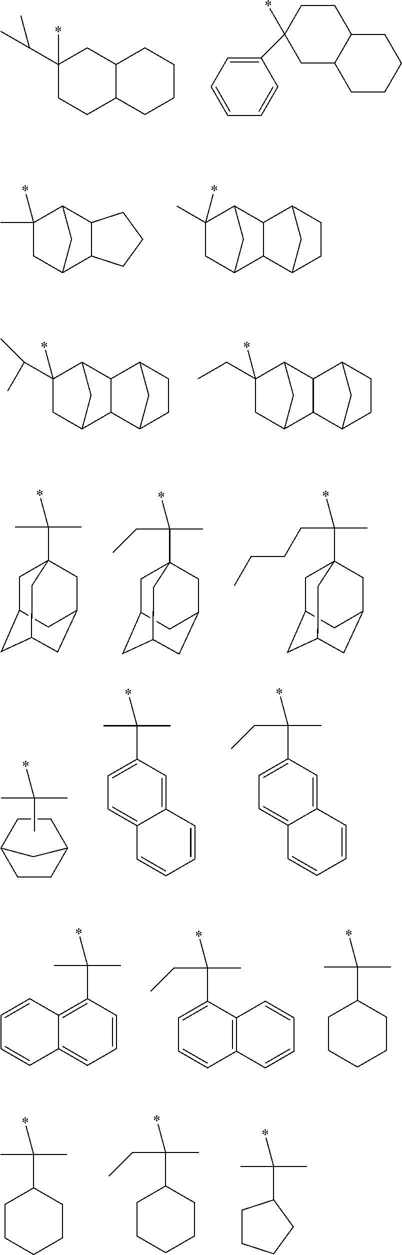

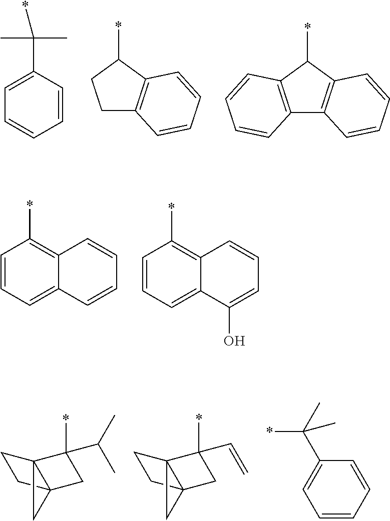

In the above structures, n may be, e.g., an integer ranging from 1 to 11, and * is a bonding site.

In an implementation, in Formula 1, R² and R³ may have the same structure. In an implementation, in Formula 1, R² and R³ may have different structures. In an implementation, in Formula 1, m1 may be 0, and 1≤m2≤2 (e.g., m2 may be 1 or 2).

In an implementation, in Formula 1, the unsubstituted C1 to C10 alkyl group of R² and R³ may include, e.g., a methyl group, an ethyl group, an n-propyl group, an isopropyl group, an n-butyl group, or a t-butyl group.

In an implementation, in Formula 1, the C1 to C10 alkyl group substituted with the F atom, of R² and R³, may include, e.g., a fluoromethyl group, a difluoromethyl group, a trifluoromethyl group, a 2,2,2-trifluoroethyl group, a pentafluoroethyl group, a heptafluoropropyl group, a perfluorobutyl group, a perfluoropentyl group, a perfluorohexyl group, a perfluoroheptyl group, a perfluorooctyl group, a perfluorononyl group, or a perfluorodecyl group.

In an implementation, in Formula 1, the C1 to C10 aryl group of R² and R³ may include, e.g., a phenyl group, a tolyl group, a dimethylphenyl group, or a naphthyl group.

In an implementation, in Formula 1, the C1 to C10 aryl group substituted with the F atom, of R² and R³, may include, e.g., a 2-fluorophenyl group, a 3-fluorophenyl group, or a 4-fluorophenyl group, without being limited thereto.

In an implementation, in Formula 1, at least one of R² and R³ may include, e.g., an iodine atom, a fluorine atom, or a hydroxyl group. In this case, the sensitivity of the photo-decomposable compound according to the embodiment to an exposure light source may be improved, and thus, the photo-decomposable compound according to the embodiments may have excellent absorbance characteristics. Accordingly, in a photolithography process using a photoresist composition including the photo-decomposable compound according to the embodiments, when a partial area of a photoresist film obtained from the photoresist composition is exposed, absorbance may be increased due to the photo-decomposable compound. Thus, quantum yield for generating acids in the exposed area may be increased.

In an implementation, the photo-decomposable compound may be represented by, e.g., Formula 1-1.

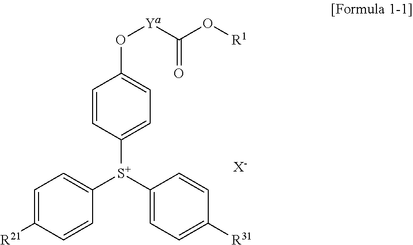

In Formula 1-1, R¹, Y^{a}, and X⁻ may be defined the same as those of Formula 1. R²¹ and R³¹ may each independently include, e.g., an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom.

In an implementation, the photo-decomposable compound may be represented by, e.g., Formula 1-2.

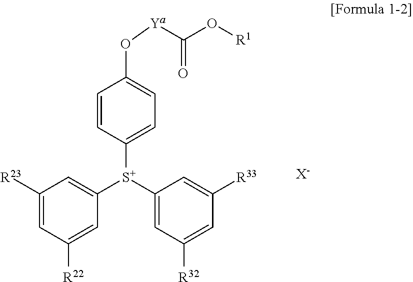

R¹, Y^{a}, and X⁻ may be defined the same as those of Formula 1. R²², R²³, R³², and R³³ may each independently include, e.g., an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom.

In an implementation, the photo-decomposable compound may be represented by, e.g., Formula 1-3.

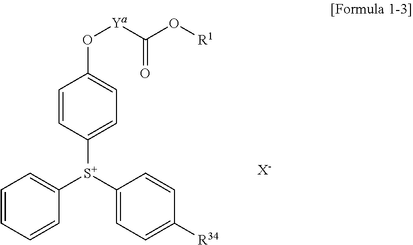

R¹, Y^{a}, and X⁻ may be defined the same as those of Formula 1. R³⁴ may be, e.g., an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom. In an implementation, R³⁴ may be bonded on Formula 1-3 at a single location or bonded at multiple locations to form a ring along with another atom included in Formula 1-3.

In an implementation, the cation component of the photo-decomposable compound according to the embodiments may have, e.g., a structure of one of Formulae 2-1 to 2-12.

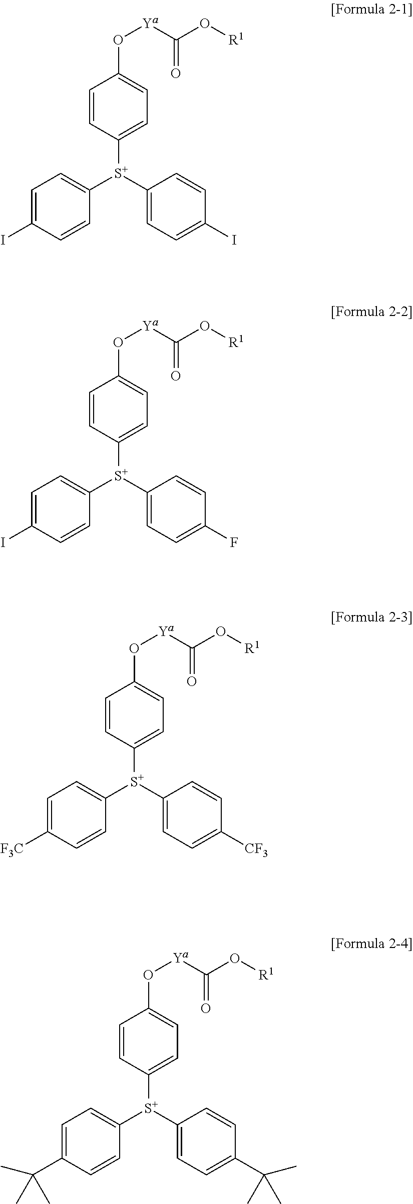

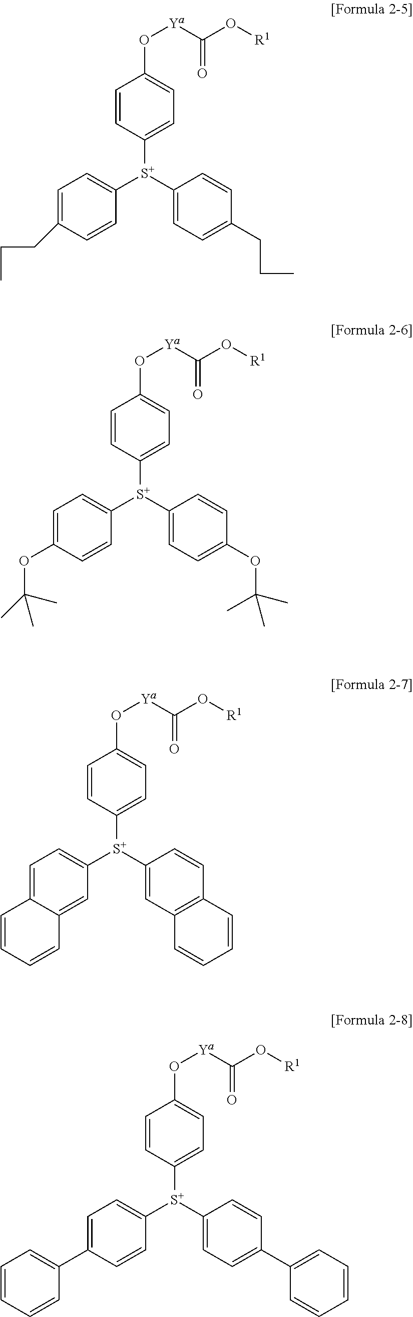

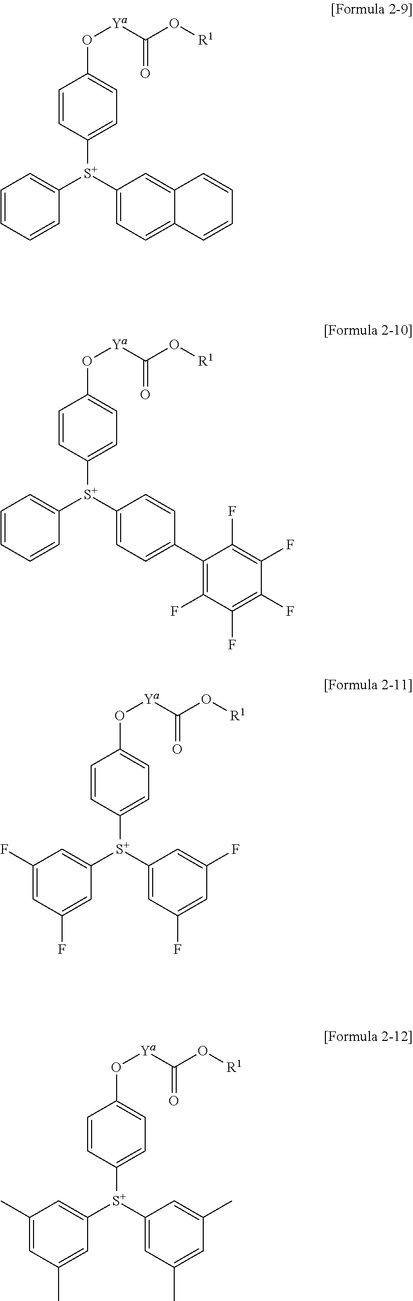

In Formulae 2-1 to 2-12, R¹ and Y^{a} may be defined the same as those of Formula 1.

In each of Formulae 1, 1-1 to 1-3, and 2-1 to 2-12, Y^{a} may be, e.g., a substituted or unsubstituted C1 to C30 alkylene group, a substituted or unsubstituted C2 to C30 alkenylene group, a substituted or unsubstituted C2 to C30 alkynylene group, a substituted or unsubstituted C7 to C30 arylenealkylene group, a substituted or unsubstituted C6 to C30 arylene group, a substituted or unsubstituted C2 to C30 heteroarylene group, a substituted or unsubstituted C2 to C30 heteroarylenealkylene group, a substituted or unsubstituted C1 to C30 alkyleneoxy group, a substituted or unsubstituted C7 to C30 arylenealkyleneoxy group, a substituted or unsubstituted C6 to C30 aryleneoxy group, a substituted or unsubstituted C2 to C30 heteroaryleneoxy group, or a substituted or unsubstituted C2 to C30 heteroarylenealkyleneoxy group.

Each of the example groups of Y^{a} may be substituted with, e.g., a halogen atom, a hydroxyl group, an isocyanate group, a glycidyl oxy group, a carboxyl group, an aldehyde group, an amino group, a siloxane group, an epoxy group, an imino group, a urethane group, an ester group, an epoxy group, an amide group, an imide group, an acrylic group, a methacryl group, a nitro group, or —HSO₃.

In an implementation, Y^{a} may be, e.g., a substituted or unsubstituted C1 to C5 alkylene group, a C5 to C20 divalent monocyclic or condensed alicyclic hydrocarbon group, or a C5 to C20 divalent monocyclic or condensed aromatic hydrocarbon group.

In an implementation, Y^{a} may be, e.g., —(CH₂)ₙ— (here, n may be an integer ranging from 1 to 5). In an implementation, Y^{a} may be, e.g., —CH₂—, —CF₂—, —CHF—, —CH₂CF₂—, or —(CH₂)_{q}(CF₂)ᵣ— (here, each of q and r may be an integer ranging from 1 to 5, and 2≤(q+r)≤6).

In an implementation, Y^{a} may be, e.g., —S—, —SO—, —SO₂—, —CO—, —O—CO—O—, —C(═O)O—, —OCO—, —CONH—, —NHCO—, or —CO—.

In an implementation, Y^{a} may be, e.g., represented by Formula 3.

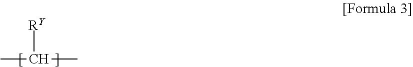

In Formula 3, R^{Y} may be or may include, e.g., a hydrogen atom, a C1 to C10 alkyl group, a C1 to C10 ether group, or a C1 to C10 alkylphenyl group. In an implementation, R^{Y} may include, e.g., one of the following structures.

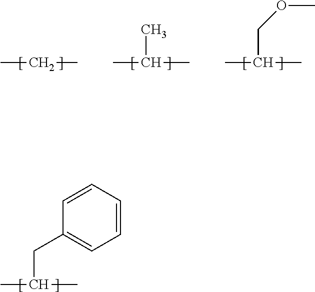

In an implementation, Y^{a} may include, e.g., one of the following structures.

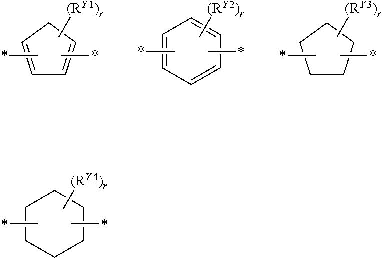

In the above, structures, * is a bonding site, r may be, e.g., an integer ranging from 0 to 2, and R^{Y1}, R^{Y2}, R^{Y3}, and R^{Y4} may each independently be, e.g., a C1 to C10 alkyl group, a cyclopropyl group, a cyclopentyl group, or a cyclohexyl group.

In Formulae 1, 1-1, 1-2, and 1-3, X⁻ may include an anion group including, e.g., SO₃⁻, CO₂⁻, or N⁻. In an implementation, X⁻ may be represented by, e.g., R^{X1}—SO₃⁻, R^{X2}—CO₂⁻, or (R^{X3}—SO₂)(R^{X4})N⁻. Here, each of R^{X1}, R^{X2}, R^{X3}, and R^{X4} may be, e.g., an organic group. The organic group may be, e.g., a substituted or unsubstituted C1 to C30 alkyl group, a substituted or unsubstituted C1 to C30 monocycloalkyl group, a substituted or unsubstituted C1 to C30 polycycloalkyl group, a partially fluorinated C1 to C30 alkyl group, or a C1 to C30 perfluoroalkyl group. R^{X3} and R^{X4} may be separate or may be bonded to each other to form a ring. In this case, R^{X3} and R^{X4} may be bonded to each other to form an alkylene group or an arylene group.

In an implementation, each of R^{X1}, R^{X2}, R^{X3}, and R^{X4} may independently include, e.g., a C1 to C5 alkyl group substituted with a fluorine atom or a fluoroalkyl group; a C1 to C5 alkylene group substituted with a fluorine atom, an iodine atom, or a fluoroalkyl group; a C5 to C20 monocyclic or condensed alicyclic hydrocarbon group substituted with a fluorine atom, an iodine atom, or a fluoroalkyl group; or a C5 to C20 monocyclic or condensed aromatic hydrocarbon group substituted with a fluorine atom, an iodine atom, or a fluoroalkyl group. In Formula 1, X⁻ may include a F atom or a fluoroalkyl group, the acidity of acid generated by light irradiation may be increased, and thus, the sensitivity of the photo-decomposable compound may be improved.

In an implementation, R^{X1}, R^{X2}, R^{X3}, and R^{X4} may each independently include, e.g., —SO₂—, —CO—, or —OC(═O)—.

In an implementation, R^{X1}, R^{X2}, R^{X3}, and R^{X4} may each independently include a monovalent or divalent linking group. In an implementation, R^{X1}, R^{X2}, R^{X3}, and R^{X4} may each independently include a divalent linking group including a carbonyl group or a divalent linking group including a sulfonyl group.

In an implementation, the photo-decomposable compound may act as a photoacid generator (PAG) that generates a relatively strong acid in the photoresist composition due to exposure. In an implementation, in Formulae 1, 1-1, 1-2, and 1-3, X⁻ may have, e.g., one of the following structures.

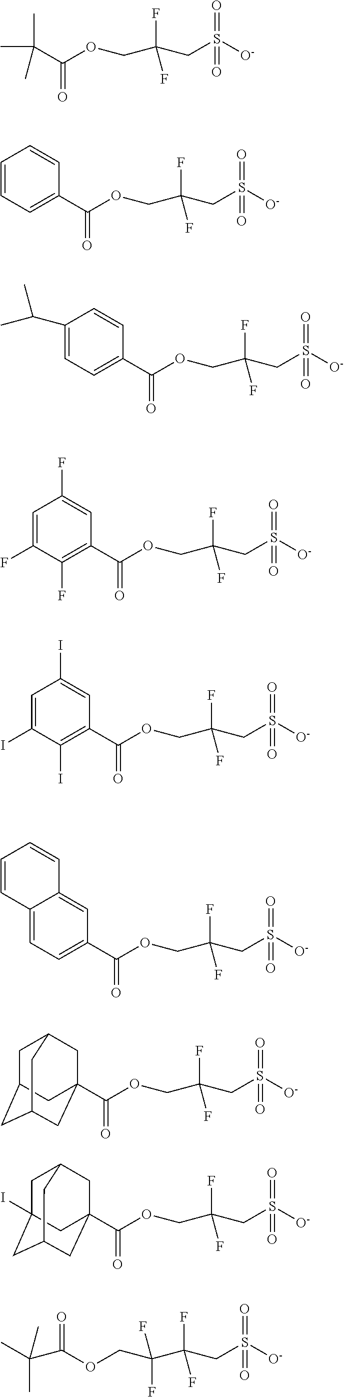

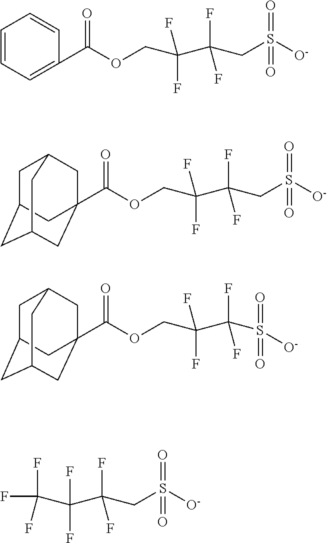 [SMILES: O=C(OCC(F)(F)C(F)(F)CS(=O)(=O)[O-])c1ccccc1 ; O=C(OCC(F)(F)C(F)(F)CS(=O)(=O)[O-])C12CC3CC(CC(C3)C1)C2 ; O=C(OCC(F)(F)C(F)(F)S(=O)(=O)[O-])C12CC3CC(CC(C3)C1)C2 ; O=S(=O)([O-])CC(F)(F)C(F)(F)C(F)(F)F]

In an implementation, the photo-decomposable compound may act as a photo-decomposable quencher (PDQ), which generates a relatively weak acid in the photoresist composition due to exposure and neutralizes acid before exposure. In an implementation, in Formulae 1, 1-1, 1-2, and 1-3, X⁻ may have, e.g., one of the following structures.

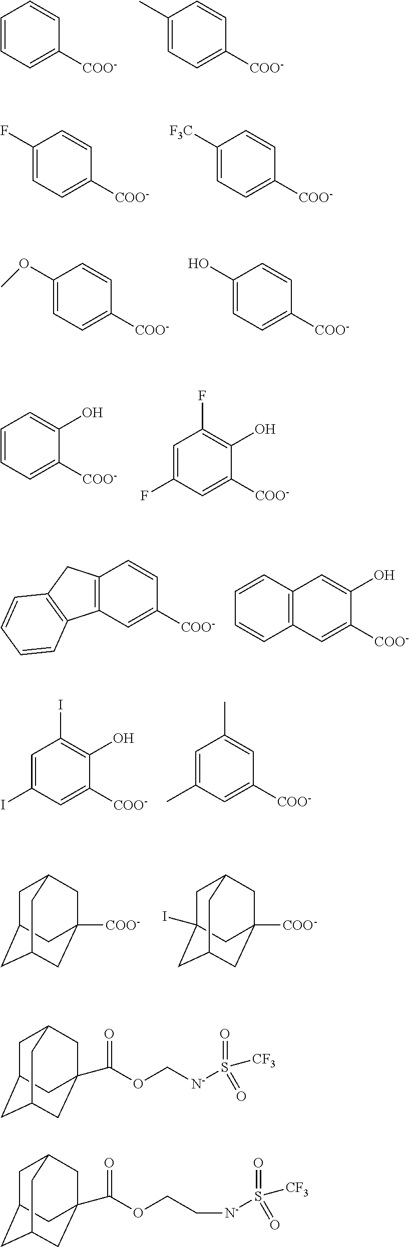

The photo-decomposable compound according to the embodiments may include a phenyl sulfonium cation component and an anion component. The phenyl sulfonium cation component may have a protecting group, which may decompose by an action of acid during exposure and may generate an alkali-soluble group. The anion component may generate acid due to exposure. The protecting group may be represented by, e.g., *—C(═O)OR (in which R may be, e.g., a substituted or unsubstituted t-butyl group and a substituted or unsubstituted C3 to C30 alicyclic group, and * is a bonding site). The protecting group may be bonded to a phenyl group of the cation component through an ether linking group (—O—).

In the photo-decomposable compound represented by Formula 1 according to the embodiments, a —O—Y^{a}—C(═O)OR¹ moiety bonded to the phenyl group of the cation component may act as an electron donating group. Accordingly, in the cation component of Formula 1 in which three phenyl groups are bonded to a sulfur atom, bond dissociation energy between a phenyl group to which the —O—Y^{a}—C(═O)OR¹ moiety is linked and the sulfur atom may be greater than bond dissociation energy between a phenyl group to which an R² group is linked and the sulfur atom and may be greater than bond dissociation energy between a phenyl group to which an R³ group is linked and the sulfur atom. Therefore, when the cation component of the photo-decomposable compound represented by Formula 1 decomposes due to acid after exposure, one phenyl group may be separated from the sulfur atom and remain as a diphenyl sulfonium byproduct. In this case, in the cation component of Formula 1, the phenyl group to which the —O—Y^{a}—C(═O)OR¹ moiety is linked may remain in the diphenyl sulfonium byproduct.

In an implementation, of the decomposition product obtained by decomposing the photo-decomposable compound of Formula 1 due to the action of acid after exposure, a relatively bulky portion may include a COO⁻ functional group, which is a hydrophilic group, and thus, solubility in a developer may be increased. Accordingly, in a photolithography process using the photoresist composition including the photo-decomposable compound represented by Formula 1, according to the embodiments, when a partial region of a photoresist film obtained from the photoresist composition is exposed, a hydrophilic group having a relatively bulky structure may be generated from the photo-decomposable compound in an exposed area of the photoresist film. As a result, a difference in solubility in a developer between the exposed area and a non-exposed area of the photoresist film may be increased, and thus, contrast may be increased. In addition, a line edge roughness (LER) and a line width roughness (LWR) may be reduced in a photoresist pattern obtained by developing the exposed photoresist film, and thus, a high pattern fidelity may be achieved. By manufacturing an integrated circuit (IC) device using a photoresist composition according to an embodiment, the dimensional precision of a pattern required for the IC device may be improved, and the productivity of a process of manufacturing the IC device may be increased.

The photoresist composition according to the embodiments may include a chemically amplified polymer, a photo-decomposable compound, and a solvent. The photo-decomposable compound may include a phenyl sulfonium cation component and an anion component. The phenyl sulfonium cation component may have a protecting group, which decomposes by an action of acid during exposure and generates an alkali-soluble group, and the anion component may generate acid due to exposure.

The photo-decomposable compound may be represented by Formula 1, and a detailed description of the photo-decomposable compound may be the same as given above. In the photoresist composition according to the embodiments, the photo-decomposable compound may be included in an amount of about 1% to about 50% by weight, based on the total weight of the chemically amplified polymer.

In the photoresist composition according to the embodiments, the chemically amplified polymer may include a polymer including a repeating unit of which solubility in a developer may be changed by the action of acid. The chemically amplified polymer may be a block copolymer or a random copolymer. In an implementation, the chemically amplified polymer may include positive-type photoresist. The positive-type photoresist may include resist for krypton fluoride (KrF) excimer laser (248 nm), resist for argon fluoride (ArF) excimer laser (193 nm), resist for fluorine (F₂) excimer laser (157 nm), or resist for extreme ultraviolet (EUV) (13.5 nm).

In an implementation, the chemically amplified polymer may include a repeating unit, which decomposes by the action of acid and increases solubility in an alkali developer. In an implementation, the chemically amplified polymer may include a repeating unit, which decomposes by the action of acid and generates phenolic acid or BrØnsted acid corresponding to the phenolic acid. In an implementation, the chemically amplified polymer may include a first repeating unit, which is derived from hydroxystyrene or derivatives thereof. The derivatives of hydroxystyrene may include hydroxystyrenes in which a hydrogen atom at an a position is substituted with a C1 to C5 alkyl group or a C1 to C5 halogenated alkyl group, or derivatives thereof. In an implementation, the first repeating unit may be derived from 3-hydroxystyrene, 4-hydroxystyrene, 5-hydroxy-2-vinylnaphtalene, or 6-hydroxy-2-vinylnaphtalene.

In an implementation, the chemically amplified polymer may have a structure in which the first repeating unit derived from hydroxystyrene or the hydroxystyrene derivative is copolymerized with at least one second repeating unit having an acid-labile protecting group. The at least one second repeating unit may include a (meth)acrylate polymer. In an implementation, the at least one second repeating unit may include polymethylmethacrylate (PMMA), poly(t-butylmethacrylate), poly(methacrylic acid), poly(norbornylmethacrylate), or a binary or ternary copolymer of repeating units of the (meth)acrylate-based polymers.

In an implementation, the chemically amplified polymer may include a blend of a first polymer having the first repeating unit and a second polymer having the at least one second repeating unit.

In an implementation, the acid-labile group, which may be included in the at least one second repeating unit, may include, e.g., tert-butoxycarbonyl (t-BOC), isonorbornyl, 2-methyl-2-adamantyl, 2-ethyl-2-adamantyl, 3-tetrahydrofuranyl, 3-oxocyclohexyl, γ-butyllactone-3-yl, mavaloniclactone, γ-butyrolactone-2-yl, 3-methyl-γ-butyrolactone-3-yl, 2-tetrahydropyranyl, 2-tetrahydrofuranyl, 2,3-propylenecarbonate yl, 1-methoxyethyl, 1-ethoxyethyl, 1-(2-methoxyethoxy)ethyl, 1-(2-acetoxyethoxy)ethyl, t-buthoxycarbonylmethyl, methoxymethyl, ethoxymethyl, trimethoxysilyl, or a triethoxysilyl group.

In an implementation, the chemically amplified polymer may further include at least one of a third repeating unit having an acrylate derivative substituent including a hydroxyl group (—OH) and a fourth repeating unit having a protecting group substituted with fluorine.

The chemically amplified polymer may have a weight-average molecular weight of about 1,000 to about 500,000. In the photoresist composition, the chemically amplified polymer may be included in an amount of about 0.1% to about 10% by weight, based on the total weight of the photoresist composition.

In an implementation, the photo-decomposable compound included in the photoresist composition may act as a PAG capable of generating acid or a PDQ capable of neutralizing acid, depending on a type of the anion component included in the photo-decomposable compound.

In an implementation, the photoresist composition according to the embodiments may further include an additional PAG configured to generate acids due to exposure.

The additional PAG may include a material having a different chemical structural formula from that of the photo-decomposable compound. In an implementation, the additional PAG may generate acid when exposed to any one of a KrF excimer laser (248 nm), an ArF excimer laser (193 nm), an F₂ excimer laser (157 nm), and an EUV laser (13.5 nm). The PAG may include a material that generates a relatively strong acid having a pKa of about −20 or more and less than about 1 due to exposure. The PAG may include, e.g., triarylsulfonium salts, diaryliodonium salts, sulfonates, or a mixture thereof. For example, the PAG may include triphenyl sulfonium triflate, triphenyl sulfonium antimonate, diphenyliodonium triflate, diphenyliodonium antimonate, methoxydiphenyliodonium triflate, di-t-butyldiphenyliodonium triflate, 2,6-dinitrobenzyl sulfonates, pyrogallol tris(alkylsulfonates), N-hydroxysuccinimide triflate, norbornene-dicarboximide-triflate, triphenyl sulfonium nonaflate, diphenyliodonium nonaflate, methoxydiphenyliodonium nonaflate, di-t-butyldiphenyliodonium nonaflate, N-hydroxysuccinimide nonaflate, norbornene-dicarboximide-nonaflate, triphenyl sulfonium perfluorobutanesulfonate, triphenyl sulfonium perfluorooctanesulfonate (PFOS), diphenyliodonium PFOS, methoxydiphenyliodonium PFOS, di-t-butyldiphenyliodonium triflate, N-hydroxysuccinimide PFOS, norbornene-dicarboximide PFOS, or a mixture thereof.

In the photoresist composition according to the embodiments, the additional PAG may be included in an amount of, e.g., about 1% to about 50% by weight, based on the total weight of the chemically amplified polymer.

In an implementation, the photoresist composition according to the embodiments may further include a basic quencher.

When acid generated by the photo-decomposable compound or the additional PAG, which is included in the photoresist composition according to the embodiments, diffuses into a non-exposed area of a photoresist film, the basic quencher may be a compound capable of trapping the acid in the non-exposed area of the photoresist film. Because the basic quencher is included in the photoresist composition according to the embodiments, a diffusion rate of the acid may be inhibited.

In an implementation, the basic quencher may include, e.g., primary aliphatic amine, secondary aliphatic amine, tertiary aliphatic amine, aromatic amine, heterocyclic ring-containing amine, a nitrogen-containing compound having a carboxyl group, a nitrogen-containing compound having a sulfonyl group, a nitrogen-containing compound having a hydroxyl group, a nitrogen-containing compound having a hydroxyphenyl group, an alcoholic nitrogen-containing compound, amides, imides, carbamates, or ammonium salts. For example, the basic quencher may include triethanol amine, triethyl amine, tributyl amine, tripropyl amine, hexamethyl disilazan, aniline, N-methylaniline, N-ethyl aniline, N-propylaniline, N,N-dimethylaniline, N,N-bis(hydroxyethyl)aniline, 2-methylaniline, 3-methylaniline, 4-methylaniline, ethylaniline, propylaniline, dimethylaniline, 2,6-diisopropylaniline, trimethylaniline, 2-nitroaniline, 3-nitroaniline, 4-nitroaniline, 2,4-dinitroaniline, 2,6-dinitroaniline, 3,5-dinitroaniline, N,N-dimethyltoluidine, or a combination thereof.

In an implementation, the basic quencher may include a photo-decomposable base. The photo-decomposable base may include a compound, which generates acid due to exposure and neutralizes the acid before exposure. The photo-decomposable base may lose ability to trap the acid when decomposed due to exposure. Accordingly, when a partial region of a photoresist film that is formed using a chemically amplified photoresist composition including the basic quencher including the photo-decomposable base is exposed, the photo-decomposable base may lose alkalinity in an exposed area of the photoresist film, while the photo-decomposable base may trap acid in a non-exposed area of the photoresist film to inhibit the diffusion of the acid from the exposed area into the non-exposed area.

The photo-decomposable base may include a carboxylate or sulfonate salt of a photo-decomposable cation. In an implementation, the photo-decomposable cation may form a complex with an anion of C1 to C20 carboxylic acid. The carboxylic acid may be, e.g., formic acid, acetic acid, propionic acid, tartaric acid, succinic acid, cyclohexylcarboxylic acid, benzoic acid, or salicylic acid.

In the photoresist composition according to the embodiments, the basic quencher may be included in an amount of, e.g., about 1% to about 50% by weight, based on the total weight of the chemically amplified polymer.

In the photoresist composition according to the embodiments, the solvent may include an organic solvent. In an implementation, the solvent may include ether, alcohol, glycolether, an aromatic hydrocarbon compound, ketone, or ester. In an implementation, the solvent may include, e.g., ethylene glycol monomethyl ether, ethylene glycol monoethyl ether, methyl cellosolve acetate, ethyl cellosolve acetate, diethylene glycol monomethyl ether, diethylene glycol monoethyl ether, propylene glycol, propylene glycol monomethyl ether, propylene glycol monomethyl ether acetate, propylene glycol monoethyl ether, propylene glycol monoethyl ether acetate, propylene glycol propyl ether acetate, propylene glycol monobutyl ether, propylene glycol monobutyl ether acetate, toluene, xylene, methyl ethyl ketone, cyclopentanone, cyclohexanone, ethyl 2-hydroxypropionate, ethyl 2-hydroxy-2-methylpropionate, ethyl ethoxyacetate, ethyl hydroxyacetate, methyl 2-hydroxy-3-methylbutanoate, methyl 3-methoxypropionate, ethyl 3-methoxypropionate, ethyl 3-ethoxypropionate, methyl 3-ethoxypropionate, methyl pyruvate, ethyl pyruvate, ethyl acetate, butyl acetate, ethyl lactoate, or butyl lactoate. The solvents may be used alone or in combination of at least two thereof. In an implementation, the amount of the solvent in the photoresist composition may be adjusted so that a solid content of the photoresist composition may range from about 0.5% to about 20% by weight.

In an implementation, the photoresist composition according to the embodiments may further include a surfactant.

The surfactant may include, e.g., fluoroalkyl benzenesulfonate, fluoroalkyl carboxylate, fluoroalkyl polyoxyethyleneether, fluoroalkyl ammonium iodide, fluoroalkyl betaine, fluoroalkyl sulfonate, diglycerin tetrakis(fluoroalkyl polyoxyethyleneether), fluoroalkyl trimethylammonium salt, fluoroalkyl aminosulfonate, polyoxyethylene nonylphenylether, polyoxyethylene octylphenylether, polyoxyethylene alkylether, polyoxyethylene laurylether, polyoxyethylene oleylether, polyoxyethylene tridecylether, polyoxyethylene cetylether, polyoxyethylene stearylether, polyoxyethylene laurate, polyoxyethylene oleate, polyoxyethylene stearate, polyoxyethylene laurylamine, sorbitan laurate, sorbitan palmitate, sorbitan stearate, sorbitan oleate, sorbitan fatty acid ester, polyoxyethylene sorbitan laurate, polyoxyethylene sorbitan palmitate, polyoxyethylene sorbitan stearate, polyoxyethylene sorbitan oleate, polyoxyethylene naphthylether, alkylbenzene sulfonate, or alkyldiphenyl etherdisulfonate. The surfactant may be included in an amount of, e.g., about 0.001% to 0.1% by weight, based on the total weight of the chemically amplified polymer.

In an implementation, the photoresist composition according to the embodiments may further include, e.g., a pigment, a preservative, an adhesion promoter, a coating aid, a plasticizer, a surface modifying agent, or a dissolution inhibitor.

The photoresist composition according to the embodiments may include a photo-decomposable compound including a phenyl sulfonium cation component and an anion component. The phenyl sulfonium cation component may have a protecting group, which may decompose by an action of acid during exposure and generate an alkali-soluble group. The anion component may generate acid due to exposure. Accordingly, in a photolithography process using the photoresist composition including the photo-decomposable compound according to the embodiments, when a partial region of a photoresist film obtained using the photoresist composition is exposed, a hydrophilic group having a relatively bulky structure may be generated from the photo-decomposable compound in an exposed area of the photoresist film. As a result, a difference in solubility in a developer between the exposed area and a non-exposed area of the photoresist film may be increased, and thus, contrast may be increased. In addition, an LER and an LWR may be reduced in a photoresist pattern obtained by developing the exposed photoresist film, and thus, a high pattern fidelity may be achieved.

Furthermore, the photo-decomposable compound included in the photoresist composition according to the embodiments may act as a photosensitizing compound having high sensitivity to a light source for exposure and excellent absorbance characteristics.

In an implementation, an EUV lithography technique may include an exposure process using EUV light having a wavelength of about 13.5 nm as an advanced technique, e.g., for superseding a lithography process using a KrF excimer laser (248 nm) and an ArF excimer laser (193 nm). An EUV lithography process may be based on a different action mechanism from the lithography process using the KrF excimer laser and the ArF excimer laser. An EUV lithography system may lack power for a light source to irradiate laser light, and there may be a limit to sufficiently increasing a dose to generate a suitable amount of acid from a PAG, from among components of a photoresist composition, during an exposure process. Thus, when an EUV lithography process is performed using other photoresist compositions including a PAG, the acid generation efficiency of the PAG could be reduced due to a relatively low dose provided by a light source of the EUV lithography system. Accordingly, it could be difficult to obtain a desired exposure sensitivity.

In the photolithography process using the photoresist composition including the photo-decomposable compound according to the embodiments, when a partial region of the photoresist film obtained from the photoresist composition is exposed, absorbance may be increased by the photo-decomposable compound according to the embodiments (e.g., the photo-decomposable compound having the structure of Formula 1), and thus, quantum yield for generating acids in the exposed area may be increased.

Therefore, when the photolithography process for manufacturing the IC device is performed using the photoresist composition according to the embodiments, light sensitivity may be increased, and sufficient dissolution contrast for a developer between the exposed area and the non-exposed area of the photoresist film may be ensured to improve resolution. Accordingly, by manufacturing an IC device using a photoresist composition according to an embodiment, the dimensional precision of a pattern required for the IC device may be improved, and the productivity of a process of manufacturing the IC device may be increased.

Hereinafter, a method of manufacturing an IC device, according to example embodiments, will be described.

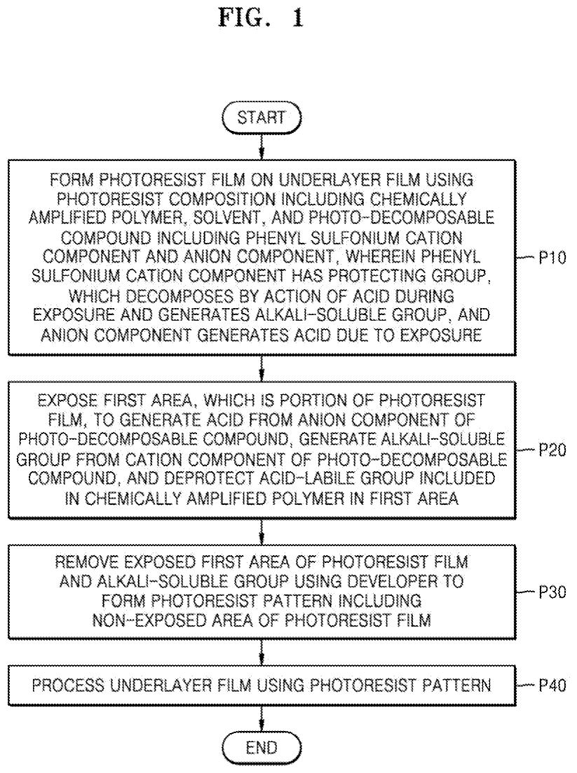

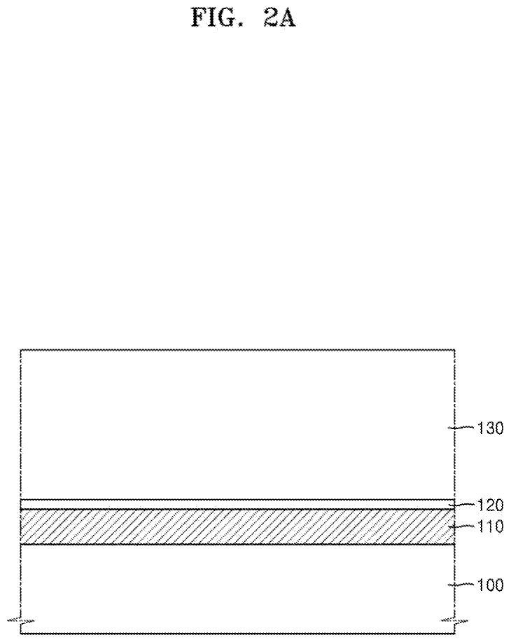

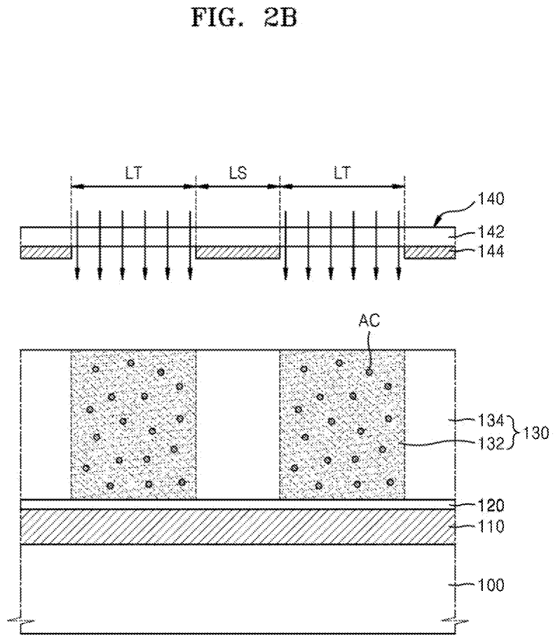

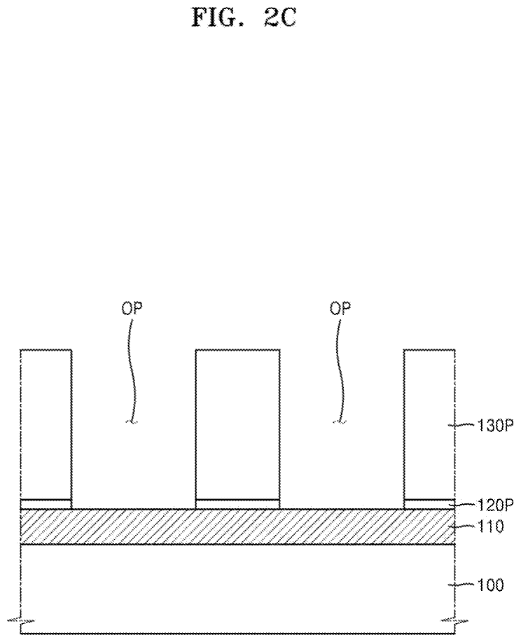

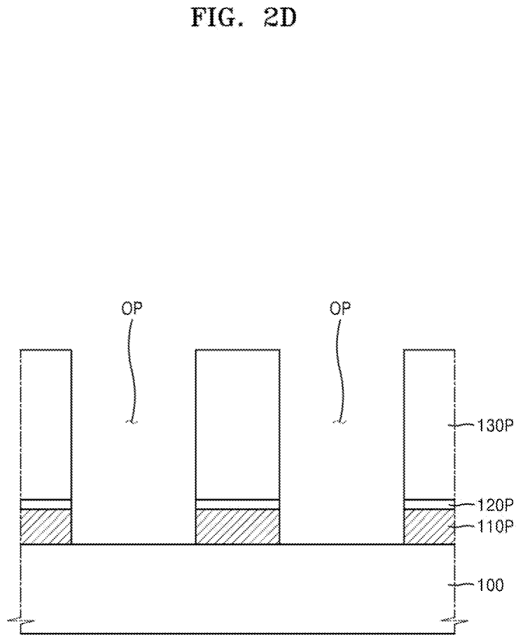

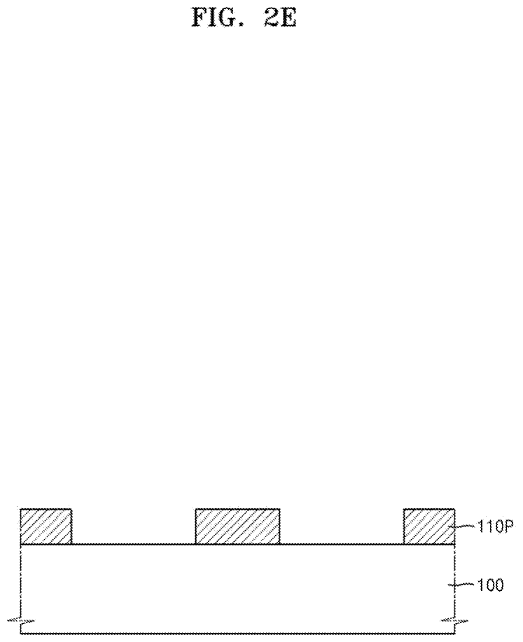

FIG. 1 is a flowchart of a method of manufacturing an IC device, according to embodiments. FIGS. 2A to 2E are cross-sectional views of stages in a method of manufacturing an IC device, according to embodiments.

Referring to FIGS. 1 and 2A, in process P10 of FIG. 1, a photoresist film 130 may be formed on an underlayer film. The underlayer film may include a substrate 100 and a feature layer 110 formed on the substrate 100.

The substrate 100 may include a semiconductor substrate. In an implementation, the substrate 100 may include an element semiconductor material (e.g., silicon (Si) or germanium (Ge)) or a compound semiconductor material (e.g., silicon germanium (SiGe), silicon carbide (SiC), gallium arsenide (GaAs), indium arsenide (InAs), or indium phosphide (InP)).

The feature layer 110 may include an insulating film, a conductive film, or a semiconductor film. In an implementation, the feature layer 110 may include a metal, an alloy, a metal carbide, a metal nitride, a metal oxynitride, a metal oxycarbide, a semiconductor, polysilicon, oxide, a nitride, oxynitride, or a combination thereof.

In an implementation, as shown in FIG. 2A, before the photoresist film 130 is formed on the feature layer 110, a developable bottom anti-reflective coating (DBARC) film 120 may be formed on the feature layer 110. In this case, the photoresist film 130 may be formed on the DBARC film 120. The DBARC film 120 may help control diffuse reflection of light from a light source used during an exposure process for manufacturing an IC device or absorb reflected light from the feature layer 110 located thereunder. In an implementation, the DBARC film 120 may include an organic anti-reflective coating (ARC) material for a krypton fluoride (KrF) excimer laser, an argon fluoride (ArF) excimer laser, or other light source. In an implementation, the DBARC film 120 may include an organic component having a light-absorbing structure. The light-absorbing structure may include, e.g., at least one benzene ring or a hydrocarbon compound in which benzene rings are fused. The DBARC film 120 may be formed to a thickness of, e.g., about 20 nm to about 100 nm. In an implementation, the DBARC film 120 may be omitted.

To form the photoresist film 130, a photoresist composition including a chemically amplified polymer, a photo-decomposable compound, and a solvent may be used. The photo-decomposable compound may include a phenyl sulfonium cation component and an anion component. The phenyl sulfonium cation component may be decomposed by an action of acid and generate an alkali-soluble group during exposure, and the anion component may generate acid due to exposure. For example, the photo-decomposable compound may be represented by Formula 1, and a detailed description of the photo-decomposable compound may be the same as that provided above.

In an implementation, the photoresist composition may further include a basic quencher. Detailed descriptions of the photo-decomposable compound according to Formula 1 and the photoresist composition may be the same as given above.

To form the photoresist film 130, the DBARC film 120 may be coated with a photoresist composition according to embodiments, and the photoresist composition may be then annealed or dried. The coating process may be performed using a method, such as a spin coating process, a spray coating process, or a dip coating process. The process of annealing the photoresist composition may be performed, e.g., at a temperature of about 80° C. to about 300° C. for about 10 seconds to about 100 seconds. A thickness of the photoresist film 130 may be several tens of times to several hundred times a thickness of the DBARC film 120. The photoresist film 130 may be formed to a thickness of, e.g., about 100 nm to about 6 μm.

Referring to FIGS. 1 and 2B, in process P20, a first area 132, which is a portion of the photoresist film 130, may be exposed, and thus, an acid AC may be generated from the anion component of the photo-decomposable compound represented by Formula 1 in the first area 132 of the photoresist film 130, an alkali-soluble group may be generated from the cation component of the photo-decomposable compound, and an acid-labile group included in the chemically amplified polymer may be deprotected. The alkali-soluble group generated from the cation component may include a diphenyl sulfonium byproduct including a phenyl group to which a —O—Y^{a}—C(═O)OR¹ group is linked, from among three phenyl groups bonded to a sulfur atom of the cation component of Formula 1. The diphenyl sulfonium byproduct may include a carboxylate anion (—COO⁻) as a hydrophilic functional group.

When an additional PAG is further included in the photoresist film 130, the acid AC may be generated from the additional PAG in the first area 132 of the photoresist film 130, which is exposed.

The alkali-soluble group that is generated from the photo-decomposable compound after the first area 132 of the photoresist film 130 is exposed may include a relatively bulky decomposition product, of the decomposition product obtained by decomposing the photo-decomposable compound by an action of the acid AC after exposure. The alkali-soluble group may include carboxylate anion (—COO—), which is the hydrophilic functional group, and thus, solubility of the exposed first area 132 in a developer may be increased. Accordingly, when the photoresist film 130 including the first area 132, which is exposed, is developed during a subsequent process, contrast may be increased by increasing a difference in solubility in a developer between an exposed area and a non-exposed area of the photoresist film 130.

In an implementation, to expose the first area 132 of the photoresist film 130, a photomask 140 having a plurality of light-shielding areas LS and a plurality of light-transmitting areas LT may be arranged at a predetermined position on the photoresist film 130, and the first area 132 of the photoresist film 130 may be exposed through the plurality of light-transmitting areas LT of the photomask 140. The first area 132 of the photoresist film 130 may be exposed using a KrF excimer laser (248 nm), an ArF excimer laser (193 nm), a fluorine (F₂) excimer laser (157 nm), or an EUV laser (13.5 nm).

The photomask 140 may include a transparent substrate 142 and a plurality of light-shielding patterns 144 on the transparent substrate 142 in the plurality of light-shielding areas LS. The transparent substrate 142 may include quartz. The plurality of light-shielding patterns 144 may include chromium (Cr). The plurality of light-transmitting areas LT may be defined by the plurality of light-shielding patterns 144.

In an implementation, an annealing or baking process may be performed to diffuse the plurality of acids AC in the first area 132 of the photoresist film 130. In an implementation, the resultant structure, which is obtained directly after the first area 132 of the photoresist film 130 is exposed in process P20 of FIG. 1, may be annealed at a temperature of about 50° C. to about 150° C. Thus, at least some of the plurality of acids AC may be diffused in the first area 132 of the photoresist film 130 so that the plurality of acids AC may be relatively uniformly distributed in the first area 132 of the photoresist film 130. The annealing process may be performed for about 10 seconds to about 100 seconds. In an implementation, the annealing process may be performed at a temperature of about 100° C. for about 60 seconds.

In an implementation, an additional annealing process may not be performed to diffuse the plurality of acids AC in the first area 132 of the photoresist film 130. In this case, in process P20 of FIG. 1, during the exposing of the first area 132 of the photoresist film 130, the plurality of acids AC may be diffused in the first area 132 of the photoresist film 130 without the additional annealing process.

As a result of the diffusion of the plurality of acids AC in the first area 132 of the photoresist film 130, the acid-labile group may be deprotected from the chemically amplified polymer included in the photoresist film 130 in the first area 132 of the photoresist film 130, and the alkali-soluble group may be generated from the cation component of the photo-decomposable compound by the action of the acids AC. The solubility of the first area 132 of the photoresist film 130 in the alkali developer may be improved due to the alkali-soluble group generated from the cation component.

When the basic quencher is included in the photoresist film 130, the basic quencher included in the photoresist film 130 in the second area 134, which is a non-exposed area, may act as a quenching base to neutralize acids that have been undesirably diffused from the first area 132 into the second area 134. In an implementation, the photo-decomposable compound included in the photoresist film 130 in the second area 134 may act as a PDQ to neutralize acids that have been undesirably diffused from the first area 132 into the second area 134.

In an implementation, in the photo-decomposable compound (e.g., the photo-decomposable compound of Formula 1) included in the photoresist film 130, when at least one of R² and R³ includes an iodine atom, a fluorine atom, or a hydroxyl group, the sensitivity of the photo-decomposable compound to an exposure light source may be improved, and thus, the photo-decomposable compound may have excellent absorbance characteristics. Accordingly, when the first area 132 of the photoresist film 130 is exposed, absorbance may be increased due to the photo-decomposable compound. Thus, quantum yield for generating acids in the first area 132, which is the exposed area, may be increased. Thus, a difference in acidity between the first area 132, which is the exposed area, and the second area 134, which is the non-exposed area, may be increased. Accordingly, a difference in solubility in a developer between the exposed area and the non-exposed area of the photoresist film 130 may be increased. As a result, a pattern having a low LER or a low LWR may be obtained in a final pattern, which is to be formed in a subsequent process.

Referring to FIGS. 1 and 2C, in process P30, the photoresist film 130 may be developed using a developer to remove the first area 132 of the photoresist film 130. As a result, a photoresist pattern 130P including the second area 134 of the photoresist film 130, which is not exposed, may be formed.

The photoresist pattern 130P may include a plurality of openings OP. After the photoresist pattern 130P is formed, portions of the DBARC film 120, which are exposed through the plurality of openings OP, may be removed to form a DBARC pattern 120P.

In an implementation, to develop the photoresist film 130, an alkali developer may be used. The alkali developer may include, e.g., a 2.38% by weight tetramethylammonium hydroxide (TMAH) solution.

In the resultant structure of FIG. 2B, the chemically amplified polymer and the compound of Formula 1 may be deprotected by the plurality of acids AC in the first area 132 of the photoresist film 130. Also, the resultant structure of FIG. 2B may include alkali-soluble groups generated from the cation component of the photo-decomposable compound. Accordingly, during the developing of the photoresist film 130 by using the developer, the solubility of the first area 132 in the developer may be improved, and thus, the first area 132 may be cleanly removed. As a result, after the photoresist film 130 is developed, residue defects, such as a bridge phenomenon and a footing phenomenon, may not occur, and the photoresist pattern 130P may have a vertical sidewall profile. As described above, by improving a profile of the photoresist pattern 130P, when the feature layer 110 is processed using the photoresist pattern 130P, a critical dimension (CD) of an intended processing region may be precisely controlled in the feature layer 110.

Referring to FIGS. 1 and 2D, in process P40, the underlayer film may be processed using the photoresist pattern 130P in the resultant structure of FIG. 2C.

To process the underlayer film, various processes, such as a process of etching the feature layer 110 exposed by the openings OP of the photoresist pattern 130P, a process of implanting impurity ions into the feature layer 110, a process of forming an additional film on the feature layer 110 through the openings OP, and a process of modifying portions of the feature layer 110 through the openings OP, may be performed. FIG. 2D illustrates a process of forming a feature pattern 110P by etching the feature layer 110, which is exposed by the openings OP, as an example of processing the underlayer film.

In an implementation, the process of forming the feature layer 110 may be omitted from the process described with reference to FIG. 2A. In this case, the substrate 100 may be processed using the photoresist pattern 130P instead of the process described with reference to the process P40 of FIG. 1 and FIG. 2D. In an implementation, various processes, such as a process of etching a portion of the substrate 100 using the photoresist pattern 130P, a process of implanting impurity ions into a partial region of the substrate 100, a process of forming an additional film on the substrate 100 through the openings OP, and a process of modifying portions of the substrate 100 through the openings OP, may be performed.

Referring to FIG. 2E, the photoresist pattern 130P and the DBARC pattern 120P, which remain on the feature pattern 110P, may be removed from the resultant structure of FIG. 2D. The photoresist pattern 130P and the DBARC pattern 120P may be removed using an ashing process and a strip process.

In the method of manufacturing an IC device, according to the embodiments described with reference to FIGS. 1 and 2A to 2E, a difference in solubility in the developer between the exposed area and the non-exposed area may be increased, and thus, an LER and an LWR may be reduced in the photoresist pattern 130P obtained from the photoresist film 130 to provide a high pattern fidelity. Accordingly, when a subsequent process is performed on the feature layer 110 and/or the substrate 100 using the photoresist pattern 130P, a dimensional precision may be improved by precisely controlling CDs of processing regions or patterns to be formed in the feature layer 110 and/or the substrate 100. Furthermore, a CD distribution of patterns to be formed on the substrate 100 may be uniformly controlled, and the productivity of a process of manufacturing an IC device may be increased.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Synthesis Example

### Synthesis of Compound of Formula 10

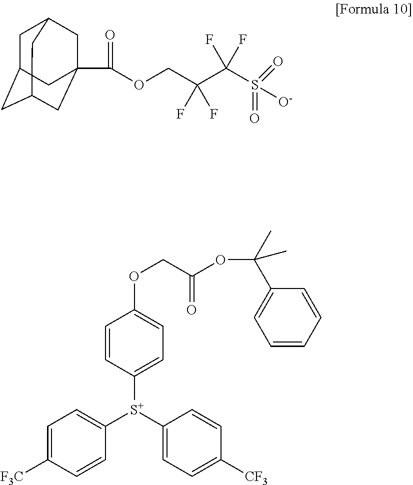 [SMILES: O=C(OCC(F)(F)C(F)(F)S(=O)(=O)[O-])C12CC3CC(CC(C3)C1)C2 ; CC(C)(OC(=O)COc1ccc([S+](c2ccc(C(F)(F)F)cc2)c2ccc(C(F)(F)F)cc2)cc1)c1ccccc1]

Initially, an intermediate product (1) (sodium 1-adamantanecarboxyl 2,2,3,3-tetrafluoro-3-sulfopropylate) was synthesized according to the following Reaction Scheme 1:

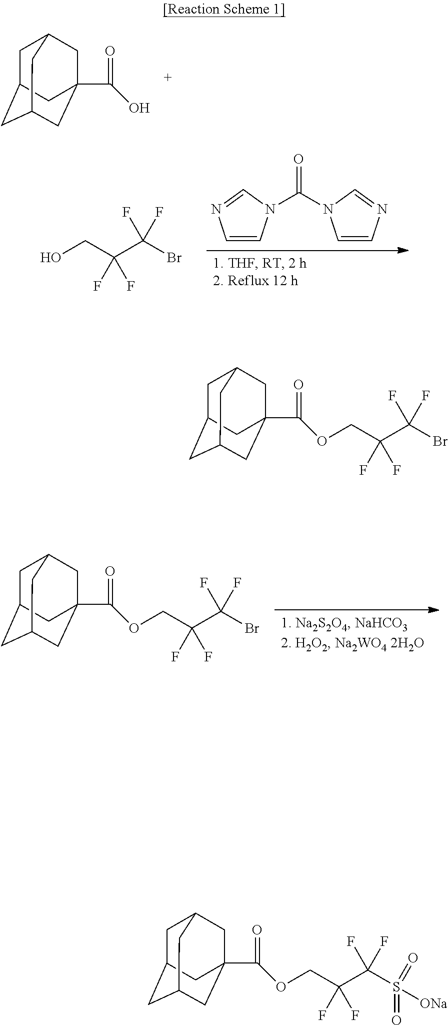

A process of Reaction Scheme 1 will now be described in detail.

1.8 g (10 mmol) of adamantyl acid was put in a 250-mL flask and dissolved in a 50 mL of a tetrahtydrofuran (THF) solvent, which was dried by nitrogen (N₂) bubbling. 1.9 g (12 mmol) of 1′, 1′-carbodiimidazole was separately dissolved in 10 mL of THF, and the obtained solution was slowly added at a rate of 10 mL/min to the previously prepared solution, followed by being stirred at room temperature for 2 hours. Afterwards, a solution prepared by separately dissolving 2.1 g (10 mmol) of 3-bromo-2,2,3,3-tetrafluoro-1-propanol in 10 mL of THF while heating and refluxing the same was slowly added at a rate of about 10 mL/min and reacted for about 12 hours. After the reaction was completed, the reacted product was cooled to room temperature, and an organic layer was collected using a separatory funnel. Afterwards, the organic layer was dried with dehydrated magnesium sulfate, filtered, and desolventized under vacuum. The obtained primary product was dissolved in 100 mL of acetonitrile and 100 mL of distilled water in a 250-mL flask, and 4.3 g (25 mmol) of sodium hydrosulfite and 3.3 g (40 mmol) of sodium bicarbonate were put in the 250-mL flask and dissolved. Thereafter, the obtained solution was heated and stirred at a temperature of about 60° C. for about 20 hours. After the reaction was completed, the reacted product was cooled to room temperature and transferred to a 250 mL flask. Thereafter, 50 mL of distilled water, 2.2 g (19 mmol) of a 30% hydrogen peroxide aqueous solution, and 10 mg (0.03 mol) of sodium tungstate dihydrate were in the 250-mL flask and stirred at room temperature for 6 hours. After the reaction was completed, an organic layer was separated and collected by adding a sodium chloride aqueous solution to the reacted product. 150 mL of diethyl ether was added to the resultant product and stirred to separate an upper organic layer again. Thereafter, the separated organic layer was dried with magnesium sulfate, filtered, and completely desolventized under vacuum to obtain 3.4 g of an intermediate product (1) (yield 85%).

Analysis values of the intermediate product (1) obtained in Reaction Scheme 1 were as follows.

¹HNMR (300 MHz, DMSO-d⁶) δ: 4.66 (t, 2H), 2.11-1.76 (m, 15H)

The intermediate product (2) (sulfonium, (4-hydroxyphenyl)bis[4-(trifluoromethyl)phenyl]-, chloride) was synthesized according to the following Reaction Scheme 2:

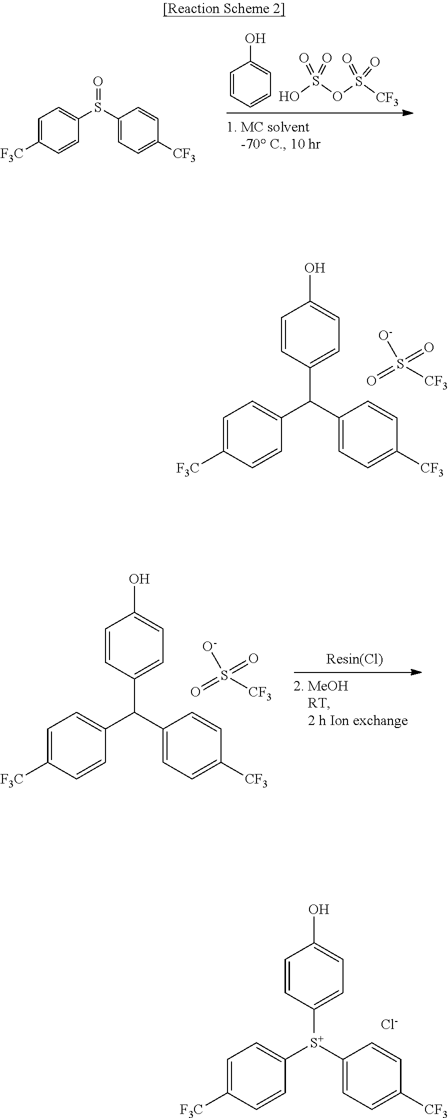

A process of Reaction Scheme 2 will now be described in detail.

3.4 g (10 mmol) of 1,1′-sulfinylbis[4-(trifluoromethyl)benzene] was dissolved in 50 mL of methylene chloride (MC) in a 3-neck flask equipped with a cooling tube and a thermometer, and the 3-neck flask was cooled to a temperature of about −70° C. Thereafter, 2.8 g (10 mmol) of trifluoromethanesulfonic anhydride and 1.1 g (12 mmol) of phenol were added and stirred for about 10 hours. A lower organic layer (MC) was separated and washed with 100 mL of distilled water three times, and residue of the obtained organic layer was filtered with dehydrated magnesium sulfate and removed. The resultant product was desolventized under vacuum to obtain 4.4 g of sulfonium, (4-hydroxyphenyl)bis[4-(trifluoromethyl)phenyl]-, triflate (yield 81%). The obtained product was ion-exchanged using an anion exchange resin in the form of C1⁻ ion in a methanol solvent for 2 hours and desolventized under vacuum to obtain 3.6 g of an intermediate product (2) (yield 99%).

Analysis values of the intermediate product (2) obtained in Reaction Scheme 2 were as follows.

¹H NMR (300 MHz, DMSO-d⁶) δ: 9.46 (s, 1H), 7.47 (d, 4H) 7.26-7.16 (m, 6H), 6.81 (d, 2H)

An intermediate product (3) was synthesized according to the following Reaction Scheme 3.

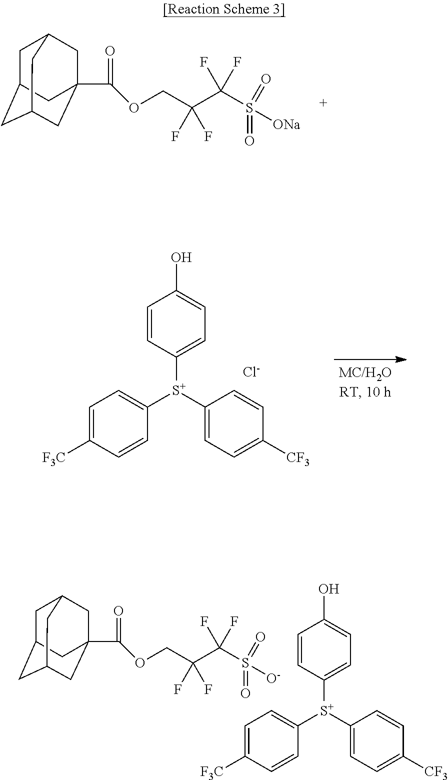

A process of Reaction Scheme 3 will now be described in detail.

4.5 g (10 mmol) of the intermediate product (1) (sodium 1-adamantanecarboxyl 2,2,3,3-tetrafluoro-3-sulfopropylate) obtained in Reaction Scheme 1 and 4.0 g of the intermediate product (2) (sulfonium, (4-hydroxyphenyl)bis[4-(trifluoromethyl)phenyl]-, chloride) obtained in Reaction Scheme 2 were put in a 250-mL flask, dissolved in 50 mL of MC and 50 mL of distilled water, and then stirred at room temperature for about 10 hours. After the reaction was completed, the underlying organic layer was separated and washed with 100 mL of distilled water three times. Next, the obtained organic layer was dried with magnesium sulfate and filtered. The resultant product was desolventized under vacuum to obtain 6.7 g of an intermediate product (3) (yield 85%).

Analysis values of the intermediate product (3) obtained in Reaction Scheme 3 were as follows.

¹H NMR (300 MHz, DMSO-d⁶) δ: 9.46 (s, 1H), 7.47 (d, 4H) 7.26-7.16 (m, 6H), 6.81 (d, 2H), 4.66 (t, 2H), 2.11-1.76 (m, 15H)

The compound of Formula 10 was synthesized according to the following Reaction Scheme 4 from the intermediate product (3) obtained in Reaction Scheme 3.

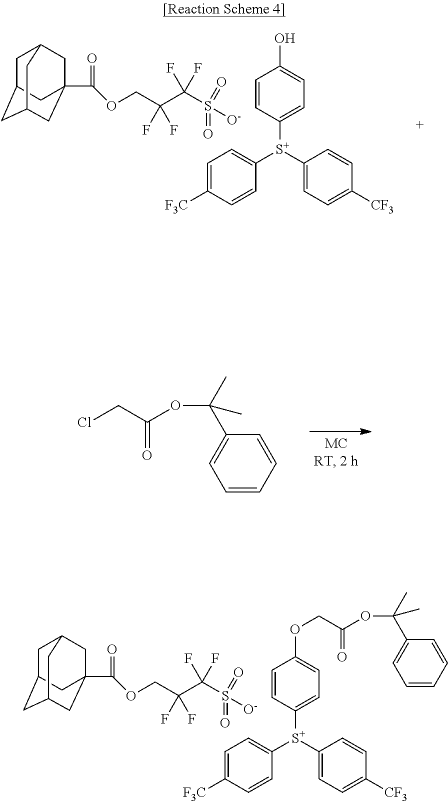 [SMILES: O=C(OCC(F)(F)C(F)(F)S(=O)(=O)[O-])C12CC3CC(CC(C3)C1)C2 ; Oc1ccc([S+](c2ccc(C(F)(F)F)cc2)c2ccc(C(F)(F)F)cc2)cc1 ; CC(C)(OC(=O)CCl)c1ccccc1 ; O=C(OCC(F)(F)C(F)(F)S(=O)(=O)[O-])C12CC3CC(CC(C3)C1)C2 ; CC(C)(OC(=O)COc1ccc([S+](c2ccc(C(F)(F)F)cc2)c2ccc(C(F)(F)F)cc2)cc1)c1ccccc1]

A process of Reaction Scheme 4 will now be described in detail.

7.9 g (10 mmol) of the intermediate product (3) obtained in Reaction Scheme 3 was dissolved in 50 mL of MC in a 250-mL flask. Thereafter, 2.3 g (12 mmol) of 1-methyl-1-phenylethyl 2-chloroacetate was put in the 250-mL flask and stirred at room temperature for 2 hours. After the reaction was completed, the reacted resultant was filtered with 100 mL of distilled water three times, and the obtained organic layer (MC) was dried with magnesium sulfate and filtered. The filtered product was desolventized under vacuum to obtain 6.3 g of a product (yield 65%).

Analysis values of the product obtained in Reaction Scheme 4 were as follows.

¹H NMR (300 MHz, DMSO-d⁶) δ: 7.54-7.17 (m, 15H), 6.96 (d, 2H), 4.99 (s, 2H) 4.66 (t, 2H), 2.11-1.76 (m, 15H), 1.54 (s, 6H)

### Evaluation Example 1

Evaluation of solubility of photo-decomposable compounds

A calculated logP (ClogP) value of each of photo-decomposable compounds of Formulae 4 to 9 according to Examples and Comparative Compounds of Comparative Formulae 1 to 6 according to Comparative Examples was evaluated, and evaluation results are shown in Table 1. Here, the ClogP value represents a concentration ratio of a non-ionized compound after exposure.

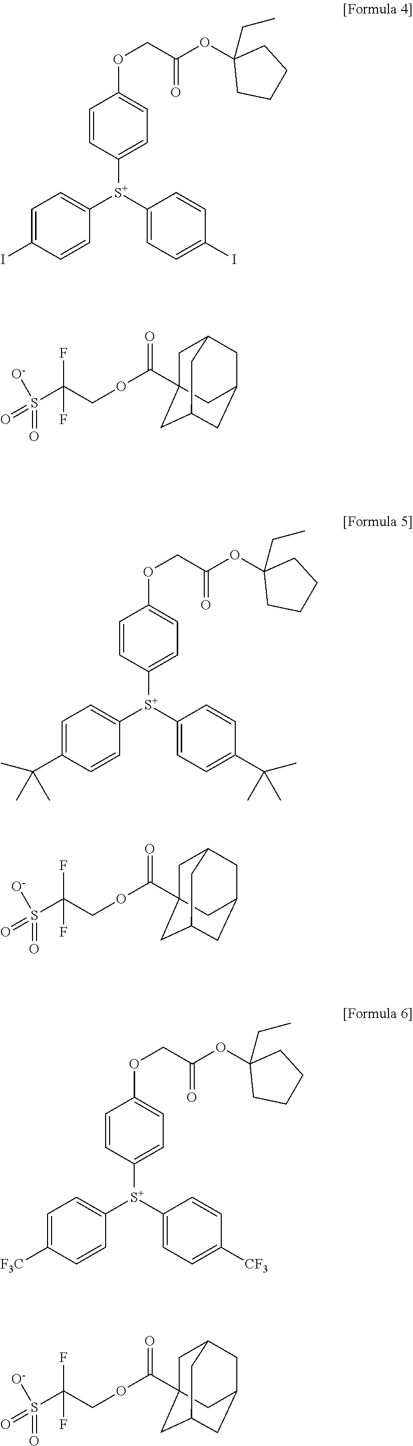 [SMILES: CCC1(OC(=O)COc2ccc([S+](c3ccc(I)cc3)c3ccc(I)cc3)cc2)CCCC1 ; O=C(OCC(F)(F)S(=O)(=O)[O-])C12CC3CC(CC(C3)C1)C2 ; CCC1(OC(=O)COc2ccc([S+](c3ccc(C(C)(C)C)cc3)c3ccc(C(C)(C)C)cc3)cc2)CCCC1 ; O=C(OCC(F)(F)S(=O)(=O)[O-])C12CC3CC(CC(C3)C1)C2 ; CCC1(OC(=O)COc2ccc([S+](c3ccc(C(F)(F)F)cc3)c3ccc(C(F)(F)F)cc3)cc2)CCCC1 ; O=C(OCC(F)(F)S(=O)(=O)[O-])C12CC3CC(CC(C3)C1)C2]

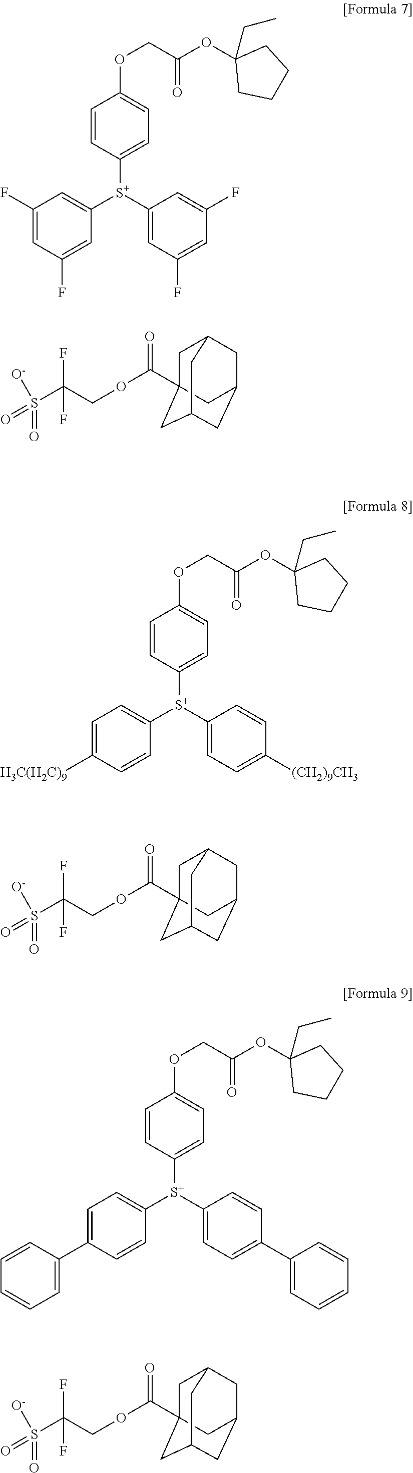

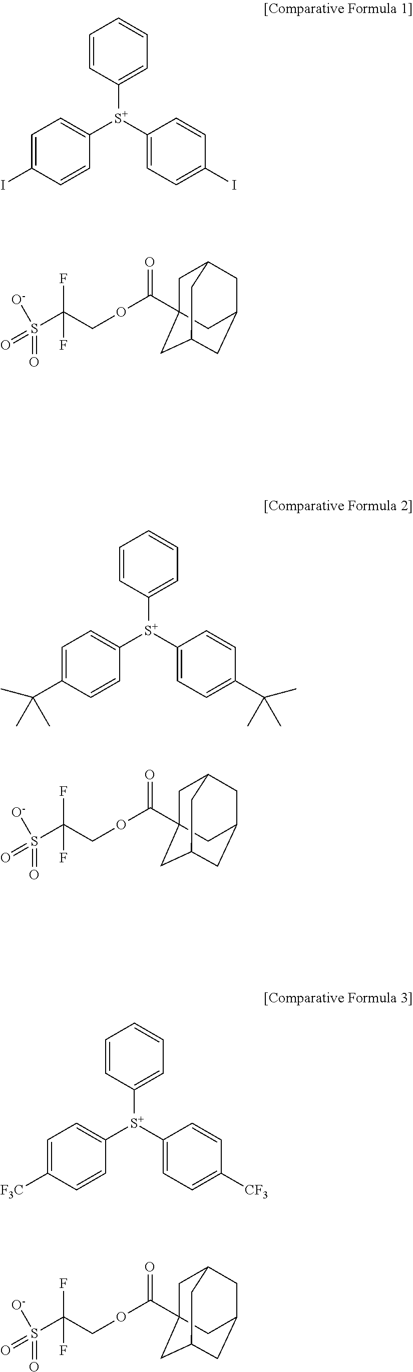 [SMILES: Ic1ccc([S+](c2ccccc2)c2ccc(I)cc2)cc1 ; O=C(OCC(F)(F)S(=O)(=O)[O-])C12CC3CC(CC(C3)C1)C2 ; CC(C)(C)c1ccc([S+](c2ccccc2)c2ccc(C(C)(C)C)cc2)cc1 ; O=C(OCC(F)(F)S(=O)(=O)[O-])C12CC3CC(CC(C3)C1)C2 ; FC(F)(F)c1ccc([S+](c2ccccc2)c2ccc(C(F)(F)F)cc2)cc1 ; O=C(OCC(F)(F)S(=O)(=O)[O-])C12CC3CC(CC(C3)C1)C2]

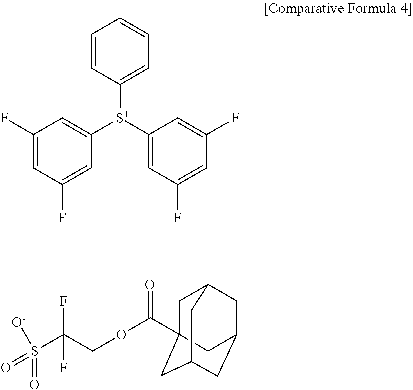 [SMILES: Fc1cc(F)cc([S+](c2ccccc2)c2cc(F)cc(F)c2)c1 ; O=C(OCC(F)(F)S(=O)(=O)[O-])C12CC3CC(CC(C3)C1)C2]

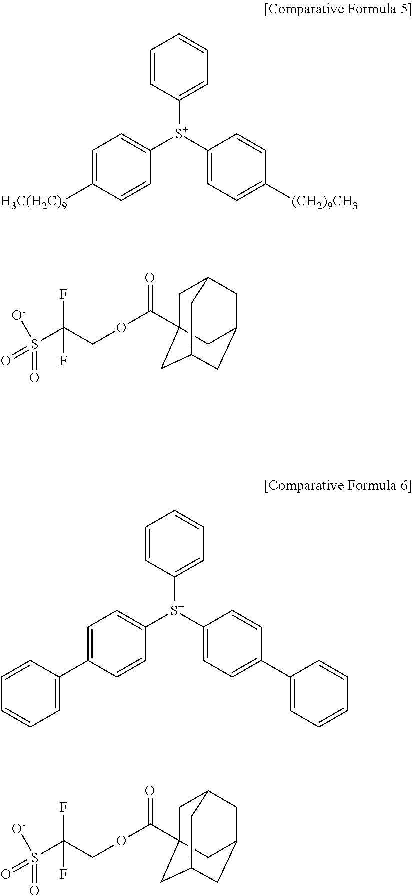

TABLE 1

| Comparative | Example | ClogP reduction |
|---|---|---|
| Compound | Compound | rate (%) of the |
| (ClogP) | (ClogP) | Examples |
| Comparative Formula 1 | Formula 4 | 28.29 |
| (1.52) | (1.09) | |
| Comparative Formula 2 | Formula 5 | 31.87 |
| (1.82) | (1.24) | |
| Comparative Formula 3 | Formula 6 | 26.76 |
| (1.42) | (1.04) | |
| Comparative Formula 4 | Formula 7 | 21.74 |
| (1.15) | (0.90) | |
| Comparative Formula 5 | Formula 8 | 40.30 |
| (3.35) | (2.00) | |
| Comparative Formula 6 | Formula 9 | 32.07 |
| (1.84) | (1.25) | |

As may be seen from the results of Table 1, the ClogP value of each of the compounds having the structures of Formulae 4 to 9 according to the Examples was about 20% to about 40% less than that of each of the compounds having the structures of Comparative Formulae 1 to 6, and had higher hydrophilicity than that of each of the compounds having the structures of Comparative Formulae 1 to 6. Accordingly, each of the compounds having the structures of Formulae 4 to 9 according to the Examples had higher solubility in a developer than each of the compounds having the structures of Comparative Formulae 1 to 6. When the compounds having the structures of Formulae 4 to 9 were exposed, a decomposition product obtained due to acid (i.e., the decomposition product including the phenyl group to which the *—O—Y^{a}—C(═O)OR¹ group was linked in Formula 1) was changed to the form of a carboxylate anions (—COO), which was a hydrophilic functional group. As a result, the hydrophilicity of the compounds having the structures of Formulae 4 to 9 were greatly increased after exposure. Accordingly, it may be seen that, in a photoresist film obtained using a photoresist composition including the compounds having the structures of Formulae 4 to 9 according to the Examples, a difference in solubility in a developer between an exposed area and a non-exposed area was increased.

### Evaluation Example 2

Evaluation of photo-decomposable compounds under harsh conditions

Photoresist films were obtained by coating a wafer with a photoresist composition including each of the compounds having the structures of Formulae 4 to 9 according to the Examples. An exposure process using an EUV laser and a developing process were performed on each of the obtained photoresist films, and thus, photoresist patterns including line-and-space patterns with a relatively high density were formed. Thereafter, pattern shapes in a normal dose region and an underdose region (or a region exposed at a smaller dose than a normal dose) of each of the obtained photoresist patterns were compared with pattern shapes in the resultant structures of Comparative Compounds 1 to 4, which were evaluated similarly to the photoresist patterns obtained according to the Examples.

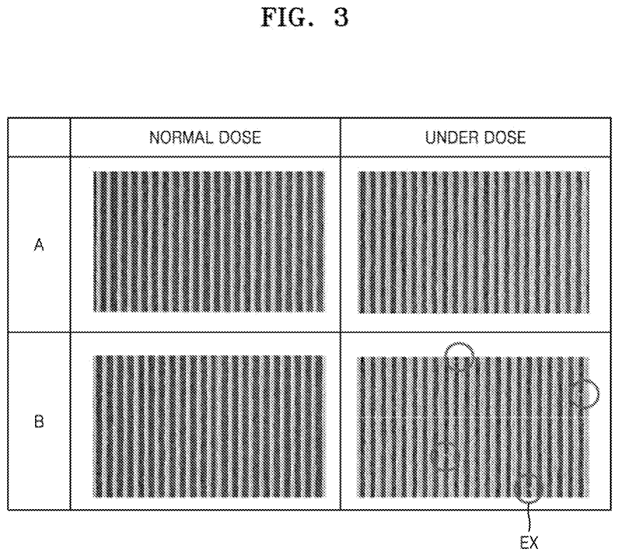

FIG. 3 shows critical dimension-scanning electron microscope (CD-SEM) images of photoresist patterns obtained using a compound according to the Examples and photoresist patterns obtained using a Comparative Compound.

In FIG. 3, part “A” shows CD-SEM images of a normal dose region and an underdose region of a result obtained using the compound of Formula 4, according to the Examples, and part “B” shows CD-SEM images of a normal dose region and an underdose region of a result obtained using the Comparative Compound of Comparative Formula 1.

As may be seen from FIG. 3, in the cases of using the Comparative Compounds, a bridge defect occurred in a photoresist pattern in portions illustrated with circles EX under harsh conditions of underdose. In contrast, in the cases of using the compounds according to the Examples, no defects occurred even under harsh conditions of underdose.

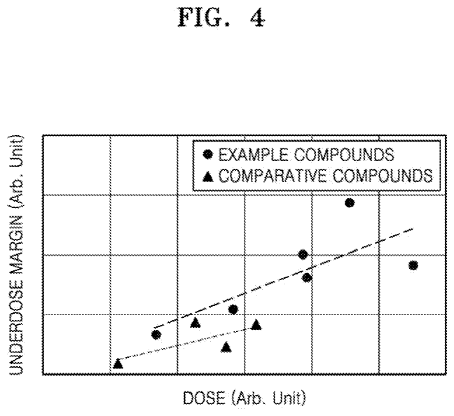

FIG. 4 is a graph showing evaluation results of underdose margins in the cases of using compounds according to Examples and the cases of using Comparative Compounds.

FIG. 4 shows comparison results of respective numerical values of underdose margins in the cases of using the compounds having the structures of Formulae 4 to 9 according to the Examples and underdose margins in the cases of using Comparative Compounds 1 to 4. From the results of FIG. 4, it may be seen that the underdose margins in the cases of using the compounds according to the Examples were superior to the underdose margins in the cases of using the Comparative Compounds.

By way of summation and review, to help improve the dimensional precision of a pattern for an IC device in a photolithography process including a positive tone development (PTD) process, a technique may increase a difference in acidity between an exposed area and a non-exposed area of a photoresist film while generating a relatively large amount of acid in the exposed area of the photoresist film with the same amount of light and also, may increase a difference in solubility between the exposed area and the non-exposed area of the photoresist film.

One or more embodiments may provide a photo-decomposable compound capable of improving solubility in a developer after exposure.

One or more embodiments may provide a photo-decomposable compound, which may help increase absorbance efficiency during exposure, may help improve contrast by increasing hydrophilicity of a decomposition product after exposure to increase a difference in solubility in a developer between an exposed area and a non-exposed area, and may help improve the dimensional precision of a pattern for an integrated circuit (IC) device.

One or more embodiments may provide a photoresist composition, which may help increase absorbance efficiency during exposure, may help improve contrast by increasing hydrophilicity of a decomposition product after exposure to increase a difference in solubility in a developer between an exposed area and a non-exposed area, and may help improve the dimensional precision of a pattern for an IC device.

One or more embodiments may provide a method of manufacturing an IC device, which may help improve productivity by improving the dimensional precision of a pattern in a photolithography process.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A photo-decomposable compound, comprising:
a phenyl sulfonium cation component; and
an anion component,
wherein:
the phenyl sulfonium cation component has a protecting group, which is decomposable by an action of acid to generate an alkali-soluble group in response to exposure,
the anion component generates acid in response to exposure,
the protecting group is represented by *—C(═O)OR, in which R is a substituted or unsubstituted t-butyl group or a substituted or unsubstituted C3 to C30 alicyclic group, and * is a bonding site, and
the protecting group is bonded to a phenyl group of the phenyl sulfonium cation component through an ether linking group.

2. The photo-decomposable compound as claimed in claim 1, wherein:
the photo-decomposable compound is represented by Formula 1: 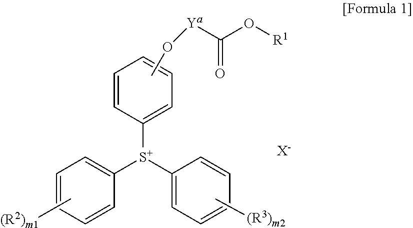
in Formula 1,
R¹ is a substituted or unsubstituted t-butyl group or a substituted or unsubstituted C3 to C30 alicyclic group,
R² and R³ are each independently a hydrogen (H) atom, an iodine (I) atom, a fluorine (F) atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom, provided that at least one of R² and R³ is an iodine (I) atom, a fluorine (F) atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom,
R² and R³ are each independently bonded on Formula 1 at a single location or bonded at multiple locations to form a ring along with another atom included in Formula 1,
m1 and m2 are each independently an integer ranging from 0 to 3, provided that at least one of m1 and m2 is 1, 2, or 3,
Y^{a} is a substituted or unsubstituted C1 to C30 divalent linear or cyclic group, and
X⁻ is a monovalent anion.

3. The photo-decomposable compound as claimed in claim 2, wherein, in Formula 1, R¹ is a substituted or unsubstituted C3 to C30 tertiary alicyclic group.

4. The photo-decomposable compound as claimed in claim 2, wherein, in Formula 1, R² and R³ have the same structure.

5. The photo-decomposable compound as claimed in claim 2, wherein, in Formula 1, R² and R³ have different structures.

6. The photo-decomposable compound as claimed in claim 2, wherein, in Formula 1, m1 is 0, and m2 is 1 or 2.

7. The photo-decomposable compound as claimed in claim 2, wherein, in Formula 1, Y^{a} is a substituted or unsubstituted C1 to C30 alkylene group, a substituted or unsubstituted C2 to C30 alkenylene group, a substituted or unsubstituted C2 to C30 alkynylene group, a substituted or unsubstituted C7 to C30 arylenealkylene group, a substituted or unsubstituted C6 to C30 arylene group, a substituted or unsubstituted C2 to C30 heteroarylene group, a substituted or unsubstituted C2 to C30 heteroarylenealkylene group, a substituted or unsubstituted C1 to C30 alkyleneoxy group, a substituted or unsubstituted C7 to C30 arylenealkyleneoxy group, a substituted or unsubstituted C6 to C30 aryleneoxy group, a substituted or unsubstituted C2 to C30 heteroaryleneoxy group, or a substituted or unsubstituted C2 to C30 heteroarylenealkyleneoxy group.

8. The photo-decomposable compound as claimed in claim 2, wherein, in Formula 1, X⁻ includes SO₃⁻, CO₂⁻, or N⁻.

9. The photo-decomposable compound as claimed in claim 1, wherein:
the photo-decomposable compound is represented by Formula 1-1: 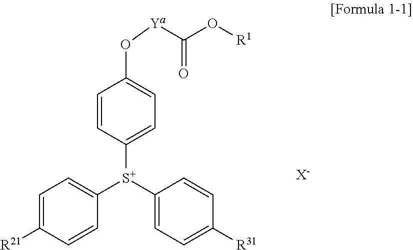
in Formula 1-1,
R¹ is a substituted or unsubstituted t-butyl group or a substituted or unsubstituted C3 to C30 alicyclic group,
R²¹ and R³¹ are each independently an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom,
R²¹ and R³¹ are each independently bonded on Formula 1-1 at a single location or bonded at multiple locations to form a ring along with another atom included in Formula 1-1,
Y^{a} is a substituted or unsubstituted C1 to C30 divalent linear or cyclic group, and
X⁻ is a monovalent anion.

10. The photo-decomposable compound as claimed in claim 1, wherein:
the photo-decomposable compound is represented by Formula 1-2: 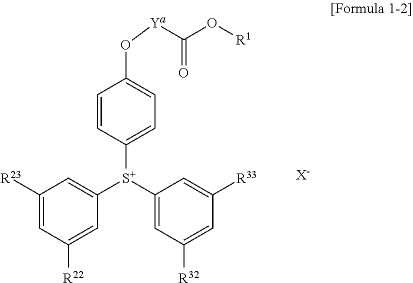
In Formula 1-2,
R¹ is a substituted or unsubstituted t-butyl group or a substituted or unsubstituted C3 to C30 alicyclic group,
R²², R²³, R³², and R³³ are each independently an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom,
Y^{a} is a substituted or unsubstituted C1 to C30 divalent linear or cyclic group, and
X⁻ is a monovalent anion.

11. The photo-decomposable compound as claimed in claim 1, wherein:
the photo-decomposable compound is represented by Formula 1-3: 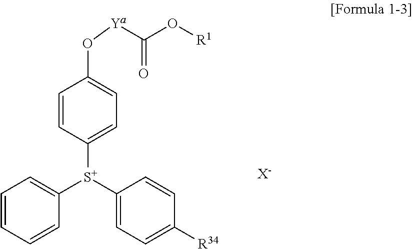
in Formula 1-3,
R¹ is a substituted or unsubstituted t-butyl group or a substituted or unsubstituted C3 to C30 alicyclic group,
R³⁴ is an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom,
R³⁴ is bonded on Formula 1-3 at a single location or is bonded at multiple locations to form a ring along with at least one atom other included in Formula 1-3,
Y^{a} is a substituted or unsubstituted C1 to C30 divalent linear or cyclic group, and
X⁻ is a monovalent anion.

12. A photoresist composition, comprising:
a chemically amplified polymer;
a photo-decomposable compound including a phenyl sulfonium cation component and an anion component, the phenyl sulfonium cation component having a protecting group that is decomposable by an action of acid in response to exposure and generates an alkali-soluble group in response to exposure, and the anion component generating an acid in response to exposure; and
a solvent,
wherein:
the photo-decomposable compound is represented by Formula 1: 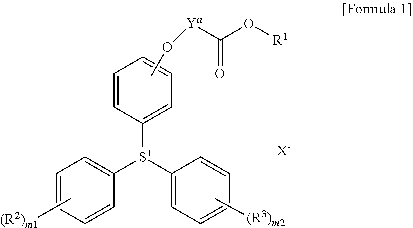
in Formula 1,
R¹ is a substituted or unsubstituted t-butyl group or a substituted or unsubstituted C3 to C30 alicyclic group,
R² and R³ are each independently a hydrogen (H) atom, an iodine (I) atom, a fluorine (F) atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom, provided that at least one of R² and R³ is an iodine (I) atom, a fluorine (F) atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom,
R² and R³ are each independently bonded on Formula 1 at a single location or bonded at multiple locations to form a ring along with another atom included in Formula 1,
m1 and m2 are each independently an integer ranging from 0 to 3, provided that at least one of m1 and m2 is 1, 2, or 3,
Y^{a} is a substituted or unsubstituted C1 to C30 divalent linear or cyclic group, and
X⁻ is a monovalent anion.

13. The photoresist composition as claimed in claim 12, wherein, in Formula 1,
R¹ is a substituted or unsubstituted C3 to C30 tertiary alicyclic group,
Y^{a} is a substituted or unsubstituted C1 to C30 alkylene group,
m2 is 1 or 2,
R³ is an iodine atom, a fluorine atom, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom.

14. The photoresist composition as claimed in claim 12, wherein:
the photo-decomposable compound has a structure of Formula 1-1: 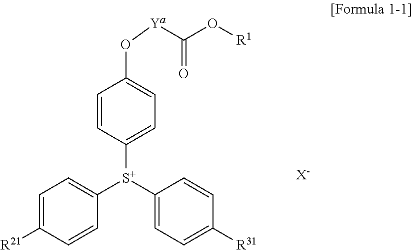
in Formula 1-1,
R²¹ and R³¹ are each independently an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom,
R²¹ and R³¹ are each independently bonded on Formula 1-1 at a single location or bonded at multiple locations to form a ring along with another atom included in Formula 1-1, and
R¹, Y^{a}, and X⁻ are defined the same as those of Formula 1.

15. The photoresist composition as claimed in claim 12, wherein:
the photo-decomposable compound has a structure of Formula 1-2: 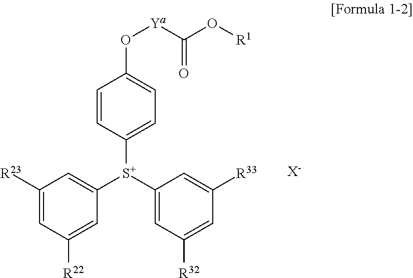
in Formula 1-2,
R²², R²³, R³², and R³³ are each independently an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom, and
R¹, Y^{a}, and X⁻ are defined the same as those of Formula 1.

16. The photoresist composition as claimed in claim 12, wherein:
the photo-decomposable compound has a structure of Formula 1-3: 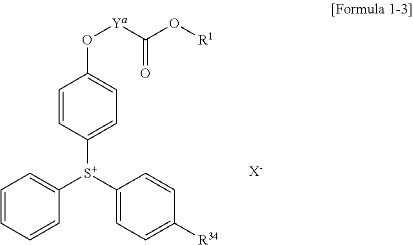
in Formula 1-3,
R³⁴ is an iodine atom, a fluorine atom, a hydroxyl group, an unsubstituted C1 to C10 alkyl group, a C1 to C10 alkyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom,
R³⁴ is bonded on Formula 1-3 at a single location or is bonded at multiple locations to form a ring along with at least one atom other included in Formula 1-3, and
R¹, Y^{a}, and X⁻ are defined the same as those of Formula 1.

17. The photoresist composition as claimed in claim 12, wherein, in Formula 1, X⁻ includes SO₃⁻, CO₂⁻, or N⁻.

18. A method of manufacturing an integrated circuit (IC) device, the method comprising:
forming a photoresist film on a underlayer film by using a photoresist composition that includes a chemically amplified polymer, a photo-decomposable compound, and a solvent, the photo-decomposable compound including a phenyl sulfonium cation component and an anion component, the phenyl sulfonium cation component having a protecting group that is decomposable by an action of acid in response to exposure and generates an alkali-soluble group in response to exposure, and the anion component generating acid in response to exposure, the protecting group being represented by *—C(═O)OR, in which R is a substituted or unsubstituted t-butyl group or a substituted or unsubstituted C3 to C30 alicyclic group, and * is a bonding site, and the protecting group being bonded to a phenyl group of the phenyl sulfonium cation component through an ether linking group,
exposing a first area, which is a portion of the photoresist film, to generate acid from the anion component of the photo-decomposable compound, deprotect an acid-labile group included in the chemically amplified polymer, and generate an alkali-soluble group from the phenyl sulfonium cation component of the photo-decomposable compound in the first area;
removing the exposed first area of the photoresist film and the alkali-soluble group using a developer to form a photoresist pattern, the photoresist pattern comprising a non-exposed area of the photoresist film, and
processing the underlayer film using the photoresist pattern.

19. The method as claimed in claim 18, wherein:
the photo-decomposable compound is represented by Formula 1: 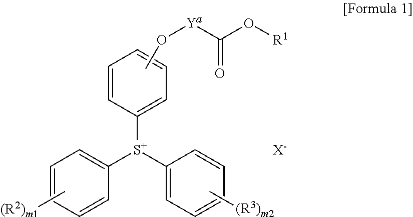
in Formula 1,
R¹ is a substituted or unsubstituted t-butyl group or a substituted or unsubstituted C3 to C30 alicyclic group,
R² and R³ are each independently a hydrogen (H) atom, an iodine (I) atom, a fluorine (F) atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom, provided that at least one of R² and R³ is an iodine (I) atom, a fluorine (F) atom, a hydroxyl group, an unsubstituted C1 to C10 alkyl group, a C1 to C10 alkyl group substituted with a fluorine atom, an unsubstituted C2 to C10 alkenyl group, a C2 to C10 alkenyl group substituted with a fluorine atom, an unsubstituted C2 to C10 alkynyl group, a C2 to C10 alkynyl group substituted with a fluorine atom, an unsubstituted C1 to C10 alkoxy group, a C1 to C10 alkoxy group substituted with a fluorine atom, an unsubstituted C6 to C20 aryl group, a C6 to C20 aryl group substituted with a fluorine atom, an unsubstituted C6 to C20 aralkyl group, or a C6 to C20 aralkyl group substituted with a fluorine atom,
R² and R³ are each independently separate or form a ring along with an atom included in Formula 1,
m1 and m2 are each independently an integer ranging from 0 to 3, provided that at least one of m1 and m2 is 1, 2, or 3,
Y^{a} is a substituted or unsubstituted C1 to C30 divalent linear or cyclic group, and
X₋ is a monovalent anion.

20. The method as claimed in claim 18, wherein exposing the first area includes exposing the first area with an extreme ultraviolet (EUV) laser.
